SEQUENCE LISTING (12 sequences)

SEQ ID NO: 1 - protein, 5 aa, synthetic construct
  REGION 1..5: note = HCDR1
NYGLS

SEQ ID NO: 2 - protein, 17 aa, synthetic construct
  REGION 1..17: note = HCDR2
WISANNGDTNYGQEFQG

SEQ ID NO: 3 - protein, 13 aa, synthetic construct
  REGION 1..13: note = HCDR3
DSSSSWARWFFDL

SEQ ID NO: 4 - protein, 11 aa, synthetic construct
  REGION 1..11: note = LCDR1
GGNIIGSKLVH

SEQ ID NO: 5 - protein, 7 aa, synthetic construct
  REGION 1..7: note = LCDR2
DDGDRPS

SEQ ID NO: 6 - protein, 11 aa, synthetic construct
  REGION 1..11: note = LCDR3
QVWDTGSDPVV

SEQ ID NO: 7 - DNA, 366 bp, synthetic construct
  misc_feature 1..366: note = cDNA heavy chain variable domain
  source 1..366: mol_type = other DNA
caggtccagctggtgcagtctggggctgaggtgaagaagcctggggcctcagtgaaggtctcctgcaaggcttctggttacacctttacaaattatggtctcagctgggtgcgacaggcccctggacaagggcttgagtggatgggatggatcagcgctaataatggcgacacaaattatggacaggaattccagggcagagtcaccatgaccacagatacatccacgagcacagcctacatggagttgaggagcctgagatctgacgacacggccgtttattactgtgcgagagactccagcagcagctgggcccgctggtttttcgatctctggggccgggggacactggtcaccgtctcctca

SEQ ID NO: 8 - protein, 122 aa, synthetic construct
  REGION 1..122: note = Polypeptide sequence heavy chain variable region
QVQLVQSGAEVKKPGASVKVSCKASGYTFTNYGLSWVRQAPGQGLEWMGWISANNGDTNYGQEFQGRVTMTTDTSTSTAYMELRSLRSDDTAVYYCARDSSSSWARWFFDLWGRGTLVTVSS

SEQ ID NO: 9 - DNA, 327 bp, synthetic construct
  misc_feature 1..327: note = cDNA light chain variable domain
  source 1..327: mol_type = other DNA
tcctatgtgctgactcagccaccctcggtgtcagtggccccaggaaagacggccaggattacctgtgggggaaacatcattggaagtaaacttgtacactggtaccagcagaagccaggccaggcccctgtgctggtcatctatgatgatggcgaccggccctcagggatccctgagcgattctctggctccaactctgggaacacggccaccctgaccatcagcagggtcgaggccggggatgaggccgactattattgtcaggtgtgggatactggtagtgatcccgtggtattcggcggagggaccaagctgaccgtcctaggt

SEQ ID NO: 10 - protein, 108 aa, synthetic construct
  REGION 1..108: note = Polypeptide sequence light chain variable region
SYVLTQPPSVSVAPGKTARITCGGNIIGSKLVHWYQQKPGQAPVLVIYDDGDRPSGIPERFSGSNSGNTATLTISRVEAGDEADYYCQVWDTGSDPVVFGGGTKLTVL

SEQ ID NO: 11 - protein, 449 aa, synthetic construct
  REGION 1..449: note = Heavy chain
QVQLVQSGAEVKKPGASVKVSCKASGYTFTNYGLSWVRQAPGQGLEWMGWISANNGDTNYGQEFQGRVTMTTDTSTSTAYMELRSLRSDDTAVYYCARDSSSSWARWFFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 214 aa, synthetic construct
  REGION 1..214: note = Light chain
SYVLTQPPSVSVAPGKTARITCGGNIIGSKLVHWYQQKPGQAPVLVIYDDGDRPSGIPERFSGSNSGNTATLTISRVEAGDEADYYCQVWDTGSDPVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

(19) United States
(12) Patent Application Publication
(10) Pub. No.: US 2024/0400669 A1
(43) Pub. Date: Dec. 5, 2024

(54) METHODS FOR TREATING ATOPIC DERMATITIS AND RELATED DISORDERS

(71) Applicant: MedImmune Limited, Cambridge (GB)

(72) Inventor: Thomas MARK, Ballerup (DK)

(21) Appl. No.: 18/695,836

(22) PCT Filed: Sep. 28, 2022
(86) PCT No.: PCT/EP2022/076955

§ 371 Date: Mar. 27, 2024

Related U.S. Application Data
(60) Provisional application No. 63/249,210, filed on Sep. 28, 2021.

(51) Int. Cl. C07K 16/24; A61K 39/00

(52) U.S. Cl. CPC C07K 16/244; A61K 2039/505; A61K 2039/545; C07K 2317/76; C07K 2317/92

(57) ABSTRACT

The present invention relates to methods for treating atopic dermatitis in a subject using an interleukin-13 (IL-13) binding protein, such as an anti-IL-13 antibody (e.g. Tralokinumab) or an IL-13-binding fragment thereof.

### FIELD OF THE INVENTION

The present invention relates to methods for treating atopic dermatitis in a subject using an interleukin-13 (IL-13) binding protein, such as an anti-IL-13 antibody or an IL-13-binding fragment thereof.

### BACKGROUND TO THE INVENTION

Atopic dermatitis (AD) is a heterogeneous inflammatory skin disease arising from genetic and environmental factors that disrupt skin barrier function and immune response (Leung, D. Y. and Guttman-Yassky, E. Deciphering the complexities of atopic dermatitis: shifting paradigms in treatment approaches. J Allergy Clin Immunol. 2014; 134: 769-779). Current management generally involves treatment combinations to suppress inflammation, restore skin barrier function, and prevent superinfection (Wollenberg, A., Oranje, A., Deleuran, M., Simon, D., Szalai, Z., Kunz, B. et al. ETFAD/EADV Eczema task force 2015 position paper on diagnosis and treatment of atopic dermatitis in adult and paediatric patients. J Eur Acad Dermatol Venereol. 2016; 30: 729-747).

Topical corticosteroids (TCSs) are overwhelmingly the most frequently prescribed class of drugs for AD patients, although long-term application of a TCS is not recommended. Topical calcineurin inhibitors (TCI) are generally effective and safe as short-term treatments. Skin malignancies and increased risk of lymphomas have prompted regulatory authorities to require a warning regarding the long-term safety of topical tacrolimus and pimecrolimus in their prescribing information, for example. First generation antihistamines are widely prescribed for acute symptomatic treatment of pruritus (itching), although their effectiveness is limited and largely attributed to their sedating effect. Oral immunosuppressants and glucocorticoids are effective, but are sometimes associated with severe toxicity and side effects, thus limiting their use to short term and/or intermittent therapy.

Cyclosporine A (CSA), a therapy for severe AD in some territories, is an immunosuppressant affecting both humoral and cellular immune responses, which increases susceptibility to infections and decreases cancer immunosurveillance. Other commonly recognized toxicities include hypertension and impaired renal and hepatic function. In addition, CSA interacts with other commonly used drugs, potentially affecting their metabolism and effect. Systemic immunosuppressants are typically reserved for treatment of moderate-to-severe AD because of their associated with adverse events and unsuitability for long-term use (Wollenberg, A., Oranje, A., Deleuran, M., Simon, D., Szalai, Z., Kunz, B. et al. ETFAD/EADV Eczema task force 2015 position paper on diagnosis and treatment of atopic dermatitis in adult and paediatric patients. J Eur Acad Dermatol Venereol. 2016; 30: 729-747). Therefore, more effective and well-tolerated therapies are required that target the mechanisms of AD pathophysiology rather than simply providing symptom relief.

A key feature of AD is upregulation of IL-13 and interleukin-4 (IL-4) in lesional and nonlesional skin, suggesting both cytokines can contribute to AD pathogenesis (see Nomura, I., Goleva, E., Howell, M. D., Hamid, Q. A., Ong, P. Y., Hall, C. F. et al. Cytokine milieu of atopic dermatitis, as compared to psoriasis, skin prevents induction of innate immune response genes. J Immunol. 2003; 171: 3262-3269; Tazawa, T., Sugiura, H., Sugiura, Y., and Uehara, M. Relative importance of IL-4 and IL-13 in lesional skin of atopic dermatitis. Arch Dermatol Res. 2004; 295: 459-464). Moreover, AD severity is associated with increased IL-13 and associated chemokine mRNA and serum levels, whereas reductions in IL-13 concentrations have correlated with treatment response and improved clinical outcomes. Although treatment with dupilumab, a human mAb that inhibits both IL-4 and IL-13 signaling, has demonstrated improvements in AD symptoms, the relative contribution of each of these cytokines to AD pathogenesis is unclear.

IL-13 is a 114 amino acid cytokine with an unmodified molecular mass of approximately 12 kDa. IL-13 is most closely related to IL-4 with which it shares 30% sequence homology at the amino acid level. The human IL-13 gene is located on chromosome 5q31 adjacent to the IL-4 gene. Although initially identified as a Th2 CD4+ lymphocyte derived cytokine, IL-13 is also produced by Th1 CD4+ T-cells, CD8+ T lymphocytes NK cells, and non-T-cell populations such as mast cells, basophils, eosinophils, macrophages, monocytes and airway smooth muscle cells. IL-13 has been linked with a number of diseases, in particular, diseases which are caused by an inflammatory response. For example, administration of recombinant IL-13 to the airways of naive non-sensitised rodents was shown to cause many aspects of the asthma phenotype including airway inflammation, mucus production and airways hyper-responsiveness. A similar phenotype was observed in a transgenic mouse in which IL-13 was specifically overexpressed in the lung. In this model, more chronic exposure to IL-13 also resulted in fibrosis.

A number of genetic polymorphisms in the IL-13 gene have also been linked to allergic diseases. In particular, a variant of the IL-13 gene in which the arginine residue at amino acid 130 is substituted with glutamine (Q130R) has been associated with bronchial asthma, atopic dermatitis and raised serum IgE levels. This particular IL-13 variant is also referred to as the Q110R variant (arginine residue at amino acid 110 is substituted with glutamine) by some groups who exclude the 20 amino acid signal sequence from the amino acid count.

Tralokinumab (also known as CAT-354 and BAK502G9) is a fully human therapeutic antibody that binds to and neutralizes IL-13, including the Q130R variant (see Popovic et al. J. Mol. Biol. (2017) 429(2): 208-219; May, R. D., Monk, P. D., Cohen, E. S., Manuel, D., Dempsey, F., Davis, N. H. et al. Preclinical development of CAT-354, an IL-13 neutralizing antibody, for the treatment of severe uncontrolled asthma. Br J Pharmacol. 2012; 166: 177-193).

Tralokinumab has previously been tested in a phase 2b study of 204 adults for the treatment of AD—where patients received 45 mg, 150 mg, or 300 mg of subcutaneous tralokinumab, or placebo, every 2 weeks for 12 weeks with concomitant topical glucocorticoids—and was found to improve change from baseline in Eczema Area Severity Index (EASI) score, together with improvements in Scoring atopic dermatitis (SCORAD), Dermatology Life Quality Index (DLQI), and pruritus numeric rating scale scores, as compared to placebo (Wollenberg J. Allergy Clin. Immunol. (2019) 143(1):135-141). Tralokinumab has also been assessed in three phase 3 trials (ECZTRA 1-3) of moderate-to-severe atopic dermatitis (Wollenberg, A., et al., Tralokinumab for moderate-to-severe atopic dermatitis: results from two 52-week, randomized, double-blind, multicentre, placebo-controlled phase III trials (ECZTRA 1 and ECZTRA 2); Br J Dermatol, 2021. 184(3): p. 437-449; Silverberg, J. I., et al., Tralokinumab plus topical corticosteroids for the treatment of moderate-to-severe atopic dermatitis: results from the double-blind, randomized, multicentre, placebo-controlled phase III ECZTRA 3 trial. Br J Dermatol, 2021. 184(3): p. 450-463).

There remains a desire in the art for further and improved treatments for AD that address, for example, at least some of the concerns referred to above.

### SUMMARY OF THE INVENTION

The inventors have found that a patient's response to an IL-13 binding protein (e.g. an anti-IL13 antibody like tralokinumab) is maintained when the dosing frequency of the antibody is decreased. Numerous advantages are associated with reducing dosing frequency, for example, improved patient compliance (e.g. fewer injections), reduction in the total amount of drug required per patient, and reduced clinician involvement (if, for example, the treatment cannot be self-administered). Patients can sometimes develop antibodies to a therapeutic protein, which neutralise the therapeutic effect. This tends not to be an issue for short term use (e.g. cancer therapy), but the likelihood increases with duration of use and drug exposure. Reducing drug exposure through dosing frequency may help prevent this effect. Increased drug exposure also increases the likelihood of side effects. Reduced dosing frequency may reduce side effects. It follows that tolerability (the balance between the efficacy of a therapeutic and its side effects) can also be improved.

The inventors have identified IGA score and worst daily pruritus NRS as positive predictors of maintained long-term response to reduced dosing frequency of Tralokinumab.

Thus, in one aspect, the invention provides an anti-interleukin-13 (IL-13) antibody, or an IL-13-binding fragment thereof, for use in a method of treating atopic dermatitis (AD) in a subject, wherein the method comprises the steps of:
(a) administering one or more prior dose(s) of the anti-IL-13 antibody, or IL-13-binding fragment thereof, to the subject, wherein each prior dose is administered from 12 days to 16 days after the immediately preceding dose, until the subject achieves an Investigator's Global Assessment (IGA) score of 0 or 1 and a worst Daily Pruritus Numeric Rating Scale (NRS) of <3 (i.e. 0-2);
(b) administering a first dose of the anti-IL-13 antibody, or IL-13-binding fragment thereof, to the subject; and
(c) administering one or more secondary dose(s) of the anti-IL-13 antibody, or IL-13-binding fragment thereof, to the subject, wherein each secondary dose is administered to the subject from 25 days to 31 days after the immediately preceding dose.

In a further aspect, the invention provides a method of treating atopic dermatitis (AD) in a subject in need thereof, wherein the method comprises the steps of:
(a) administering one or more prior dose(s) of the anti-IL-13 antibody, or IL-13-binding fragment thereof, to the subject, wherein each prior dose is administered from 12 days to 16 days after the immediately preceding dose, until the subject achieves an Investigator's Global Assessment (IGA) score of 0 or 1 and a worst Daily Pruritus Numeric Rating Scale (NRS) of <3;
(b) administering a first dose of the anti-IL-13 antibody, or IL-13-binding fragment thereof, to the subject; and
(c) administering one or more secondary dose(s) of the anti-IL-13 antibody, or IL-13-binding fragment thereof, to the subject, wherein each secondary dose is administered to the subject from 25 days to 31 days after the immediately preceding dose.

In yet a further aspect, the invention provides use of an anti-IL-13 antibody, or an IL-13-binding fragment thereof, in the manufacture of a medicament for treating atopic dermatitis (AD) in a subject, wherein the method for treating AD comprises the steps of:
(a) administering one or more prior dose(s) of the anti-IL-13 antibody, or IL-13-binding fragment thereof, to the subject, wherein each prior dose is administered from 12 days to 16 days after the immediately preceding dose, until the subject achieves an Investigator's Global Assessment (IGA) score of 0 or 1 and a worst Daily Pruritus Numeric Rating Scale (NRS) of <3;
(b) administering a first dose of the anti-IL-13 antibody, or IL-13-binding fragment thereof, to the subject; and
(c) administering one or more secondary dose(s) of the anti-IL-13 antibody, or IL-13-binding fragment thereof, to the subject, wherein each secondary dose is administered to the subject from 25 days to 31 days after the immediately preceding dose.

Preferably, each prior dose of the anti-IL-13 antibody, or IL-13-binding fragment thereof, is administered about 2 weeks (e.g. 14 days) after the immediately preceding dose.

Preferably, each secondary dose of the anti-IL-13 antibody, or IL-13-binding fragment thereof, is administered about 4 weeks (e.g. 28 days) after the immediately preceding dose.

In some embodiments, the subject achieves an IGA score of 0 or 1 (e.g. an IGA score of 0 or 1) and a Worst Daily Pruritus NRS of <3 (i.e. 0-2) for at least one week, at least two consecutive weeks, at least three consecutive weeks or at least four consecutive weeks.

In some embodiments, the subject achieves a mean IGA score of 0 or 1 and a mean Worst Daily Pruritus NRS of <3 (i.e. 0-2) over a one week, two week, three week or four week period.

The method may comprise administering the one or more prior doses(s) of an anti-IL-13 antibody, or an IL-13-binding fragment thereof, to the subject for from 2 to 36 weeks. Preferably, the method comprises administering the one or more prior doses(s) of an anti-IL-13 antibody, or an IL-13-binding fragment thereof, to the subject for from 12 to 16 weeks, e.g. for about 12 weeks or for about 16 weeks.

The method may comprise administering one or more secondary dose(s) of the anti-IL-13 antibody, or IL-13-binding fragment thereof, to the subject for at least 8 weeks, at least 12 weeks, at least 16 weeks, at least 20 weeks, at least 24 weeks, at least 28 weeks, at least 32 weeks, at least 36 weeks, at least 40 weeks, at least 44 weeks, at least 48 weeks, or at least 52 weeks.

In some embodiments, an initial loading dose of the anti-IL-13 antibody, or IL-13-binding fragment thereof, is administered to the subject before administering the one or more prior doses.

Preferably, each prior dose of the anti-IL-13 antibody, or IL-13-binding fragment thereof, is 250-350 mg (e.g. 300 mg). However, in some embodiments, each prior dose may be 150 mg.

Preferably, the first dose and each secondary dose of the anti-IL-13 antibody, or IL-13-binding fragment thereof, is 250-350 mg (e.g. 300 mg). However, in some embodiments, the first dose and each secondary dose may be 150 mg.

Preferably, the initial loading dose of the anti-IL-13 antibody, or IL-13-binding fragment thereof, is 600 mg.

Preferably, each dose of the anti-IL-13 antibody, or IL-13-binding fragment thereof, is administered subcutaneously. For example, the anti-IL-13 antibody, or IL-13-binding fragment thereof, may be administered in one or two injections.

In some embodiments, the anti-IL-13 antibody, or IL-13-binding fragment thereof, is administered as a monotherapy.

In other embodiments, the anti-IL-13 antibody, or IL-13-binding fragment thereof, is administered with a second agent, e.g. a topical corticosteroid, a topical calcineurin inhibitor, an anti-histamine, an emollient, or an anti-bacterial therapeutic.

In some embodiments, the method achieves:
(i) ≥75% improvement of Eczema Area and Severity Index (EASI-75) compared to baseline; and/or
(ii) an IGA score of 0 or 1 after 12 weeks, after 16 weeks, after 20 weeks, after 24 weeks, after 28 weeks, after 32 weeks, after 36 weeks, after 40 weeks, after 44 weeks, after 48 weeks, or after 52 weeks.

In any of the method described herein, the AD may be moderate-to-severe AD.

In some embodiments, each dose of the of the anti-IL-13 antibody, or IL-13-binding fragment thereof, is administered as a pharmaceutical composition comprising 50 mM sodium acetate buffer, 85 mM sodium chloride, 0.01% (w/v) polysorbate 80, wherein the pharmaceutical composition has a pH of 5.5.

In preferred embodiments, the anti-IL-13 antibody, or IL-13-binding fragment thereof, is a monoclonal IL-13 antibody, or IL-13-binding fragment thereof, such as a human monoclonal IL-13 antibody, or IL-13-binding fragment thereof.

In some embodiments, the anti-IL-13 antibody, or IL-13-binding fragment thereof, is an IgG4 antibody.

In some embodiments, the IL-13-binding fragment is selected from a Fab, Fab′, F(ab′)2, Fd, Fv, single-chain Fv (scFv), or disulfide-linked Fvs (sdFv).

Preferably, the anti-IL-13 antibody, or IL-13-binding fragment thereof, comprises a heavy chain variable region (HCVR) and a light chain variable region (LCVR), wherein:
(i) the heavy chain variable region comprises:
   a heavy chain complementarity determining region 1 (HCDR1) comprising an amino acid sequence of SEQ ID NO:1;
   a heavy chain complementarity determining region 2 (HCDR2) comprising an amino acid sequence of SEQ ID NO:2; and
   a heavy chain complementarity determining region 3 (HCDR3) comprising an amino acid sequence of SEQ ID NO:3; and
(ii) the light chain variable region comprises:
   a light chain complementarity determining region 1 (LCDR1) comprising an amino acid sequence of SEQ ID NO:4;
   a light chain complementarity determining region 2 (LCDR2) comprising an amino acid sequence of SEQ ID NO:5; and
   a light chain complementarity determining region 3 (LCDR3) comprising an amino acid sequence of SEQ ID NO:6.

Preferably, the anti-IL-13 antibody, or the IL-13-binding fragment thereof, comprises:
(i) an amino acid sequence that is at least 80%, at least 85%, at least 90%, at least 95%, at least 96%, at least 97%, at least 98%, or at least 99% identical to a heavy chain variable region sequence of SEQ ID NO: 8; and/or
(ii) an amino acid sequence that is at least 80%, at least 85%, at least 90%, at least 95%, at least 96%, at least 97%, at least 98%, or at least 99% identical to a light chain variable region sequence of SEQ ID NO: 10.

More preferably, the anti-IL-13 antibody, or the IL-13-binding fragment thereof, comprises a heavy chain variable region sequence of SEQ ID NO: 8 and a light chain variable region sequence of SEQ ID NO: 10.

In some preferred embodiments, the anti-IL-13 antibody, or the IL-13-binding fragment thereof, comprises: (i) an amino acid sequence that is at least 80%, at least 85%, at least 90%, at least 95%, at least 96%, at least 97%, at least 98%, or at least 99% identical to the heavy chain sequence of SEQ ID NO: 11; and/or (ii) an amino acid sequence that is at least 80%, at least 85%, at least 90%, at least 95%, at least 96%, at least 97%, at least 98%, or at least 99% identical to the light chain sequence of SEQ ID NO: 12.

More preferably, the anti-IL-13 antibody, or the IL-13-binding fragment thereof, comprises a heavy chain of SEQ ID NO: 11 and a light chain sequence of SEQ ID NO: 12.

Most preferably, the anti-IL-13 antibody is Tralokinumab or an IL-13 binding fragment thereof.

### DESCRIPTION OF FIGURES

FIG. 1 shows the ECZTRA 1 and 2 trial design. AD, atopic dermatitis; EASI, Eczema Area and Severity Index; IGA, Investigator's Global Assessment; Q2W, every 2 weeks; Q4W, every 4 weeks; TCS, topical corticosteroid. N values are pooled from ECZTRA 1 and ECZTRA 2. Wollenberg A et al. Br J Dermatol 2020, doi: 10.1111/bjd.19574.

FIG. 2 shows the maintenance of week 16 IGA 0/1 and EASI-75 responses at week 52 without rescue medication following Q2W and Q4W maintenance treatment with Tralokinumab after initial treatment with Q2W Tralokinumab. Analysis of patients who achieved a clinical response of (A) IGA 0/1 at Week 16, (B) EASI-75 at Week 16, (C) IGA 0/1 or EASI-75 at Week 16 (all without rescue medication), with Tralokinumab Q2W and were re-randomized to receive either Tralokinumab Q2W, Tralokinumab Q4W, or placebo until Week 52. Patients who received rescue medication or were transferred to open-label treatment considered non-responders. Patients with missing data were imputed as non-responders. Differences in response rates were analyzed using the Cochran-Mantel-Haenszel test stratified by region and study ID. EASI, Eczema Area and Severity Index; IGA, Investigator's Global Assessment; Q2W, every 2 weeks; Q4W, every 4 weeks.

FIG. 3 shows the methods used to identify predictors for maintained response at week 52.

FIG. 4 shows the top 10 predictors identified for maintained response at week 52. ^{a}Predictors ranked by % times predictor was identified as a significant (P<0.05) predictive variable for IGA 0/1 without rescue use at Week 52 based on logistic regression. Use of rescue medication (incl TCS) considered as nonresponse and missing data imputed as nonresponse; 1000 replications.

FIG. 5 shows the maintenance of response at week 52 following Q2W and Q4W maintenance treatment with Tralokinumab when using IGA 0/1 and/or Worst Daily Pruritus NRS as predictors. Use of rescue medication (incl TCS) considered as nonresponse and missing data imputed as nonresponse. Treatment differences were estimated with a Mantel-Haenszel analysis stratified by baseline IGA, region, and trial. The comparable responder rates and P>0.05 indicates equivalence between Q2W and Q4W.

FIG. 6 shows maintenance of response at week 52 following Q2W and Q4W maintenance treatment with Tralokinumab for patients that had experienced stable disease control over a period of time when using IGA 0/1 and Worst Daily Pruritus NRS as a predictor. Use of rescue medication (incl TCS) considered as nonresponse and missing data imputed as nonresponse. Treatment differences were estimated with a Mantel-Haenszel analysis stratified by baseline IGA, region, and trial. The comparable responder rates and P>0.05 indicates equivalence between Q2W and Q4W.

FIG. 7 shows maintenance of response at week 52 following Q2W and Q4W maintenance treatment with Tralokinumab for patients that had demonstrated an early response to Q2W dosing of Tralokinumab when using IGA 0/1 and Worst Daily Pruritus NRS as a predictor. Use of rescue medication (incl TCS) considered as nonresponse and missing data imputed as nonresponse. Treatment differences were estimated with a Mantel-Haenszel analysis stratified by baseline IGA, region, and trial. The comparable responder rates and P>0.05 indicates equivalence between Q2W and Q4W.

### DETAILED DESCRIPTION

The invention relates to methods for treating atopic dermatitis in a subject using an interleukin-13 (IL-13) binding protein (e.g. an anti-IL-13 antibody or an IL-13-binding fragment thereof).

### Atopic Dermatitis

“Atopic dermatitis” (AD), as used herein, means an inflammatory skin disease characterized by intense pruritus (e.g. severe itch) and by scaly and dry eczematous lesions.

The term “atopic dermatitis” includes AD caused by or associated with epidermal barrier dysfunction, allergy (e.g. allergy to certain foods, pollen, maid, dust mite, animals, etc.), radiation exposure, and/or asthma. In some embodiments, the present invention relates to moderate-to-severe or severe AD.

As used herein, “moderate-to-severe AD” is characterized by intensely pruritic, widespread skin lesions that are often complicated by persistent bacterial, viral or fungal infections. Moderate-to-severe AD also includes chronic AD. In many cases, the chronic lesions include thickened plaques of skin, lichenification and fibrous papules. In general, patients affected by moderate-to-severe AD also have more than 20% of the body's skin affected, or 10% of skin area in addition to involvement of the eyes, hands and body folds. Moderate-to-severe AD is also considered to be present in patients who frequently require treatment with a topical corticosteroid. In the clinical studies reported herein a subject with “moderate to severe AD” was a subject having an IGA score of 3-4.

As used herein, “severe AD” refers to chronic relapsing AD that is refractory to treatment with medium-potency and high-potency TCS and/or immunosuppressant therapy. Severe AD is also characterized by chronic intensely pruritic lesions affecting more than 20% of the body surface area. Severe AD can be considered to be present in subjects with chronic AD according to the Eichenfield criteria (Eichenfield et al 2014, J. Am. Acad. Dermatol. 70: 338-351), for which treatment with a potent topical corticosteroid (TCS) is indicated, and/or where the subject is resistant to treatment with a systemic corticosteroid and/or non-steroidal immunosuppressant. In the clinical studies reported herein a subject with “severe AD” was a subject having an IGA score of 4. Thus, in certain embodiments, the method treats severe AD in a subject, where the subject has an IGA score of 4 at baseline. A subject with “severe AD” may have AD on at least 10% of their body surface area at screening and baseline, an EASI score of ≥20 at screening and baseline, and an IGA score of ≥3 at screening and baseline.

### Subject

As used herein, the term “subject” includes human and non-human animals, particularly mammals. Typically, the subject is a human, as shown in the examples below.

A subject with AD (especially moderate-to-severe AD or severe AD) may be resistant, non-responsive or inadequately responsive to treatment with a non-steroid systemic immunosuppressant. The term “non-steroid systemic immunosuppressant” includes cyclosporine A (CSA), methotrexate, mycophenolate mofetil, azathioprine, and interferon-gamma. In certain embodiments, the term also includes immunobiologics such as tumor necrosis factor alpha (TNFa) inhibitors (e.g. an anti-TNFa antibody such as infliximab), CD11a inhibitors (e.g. an anti-CD11a antibody such as efalizumab), IgE inhibitors (e.g. omalizumab), CD20 inhibitors (e.g. rituximab). Thus, in some cases, the methods described herein may treat AD in subjects that are resistant, nonresponsive (refractory) or inadequately responsive to treatment with a systemic immunosuppressant. The term “resistant, non-responsive or inadequately responsive to a systemic immunosuppressant” refers to a subject with AD that has been treated with a systemic immunosuppressant and the immunosuppressant did not have a therapeutic effect, e.g. a subject with moderate-to-severe AD or severe AD (such as those with chronic relapsing AD) that has been treated with a non-steroid systemic immunosuppressant for between 1-3 months and did not show a decrease in one or more AD-associated parameter score(s). The time for the assessment of a therapeutic effect will vary depending on the typical timeframe for onset of action of the non-steroid systemic immunosuppressant. Such timeframes are well known. For example, for cyclosporine the onset of action is typically 2-6 weeks, but for other non-steroid systemic immunosuppressants it is typically around 8-12 weeks.

In some embodiments, immunosuppressant treatment has been deemed not medically advisable by a physician for the subject. Such a subject may be identified by the following criteria: (1) no prior immunosuppressant exposure; (2) not currently a candidate for immunosuppressant treatment due to: medical contraindication(s); or hypersensitivity to the immunosuppressant or excipient(s); use of concomitant medications prohibited with immunosuppressant; or increased susceptibility to immunosuppressant induced renal damage or increased risk of serious infections; (3) previous intolerance and/or unacceptable toxicity on previous exposure to an immunosuppressant; and/or (4) requirement for immunosuppressant at doses or duration beyond that specified in the prescribing information.

In any of the methods described herein for the treatment of atopic dermatitis, e.g. severe atopic dermatitis, the subject may be one in which the atopic dermatitis is not adequately controlled by cyclosporine A (CSA), e.g. oral cyclosporine A, or the subject has contraindications to cyclosporine A (CSA), e.g. oral cyclosporine A.

An inadequate response to CSA is defined as flare of AD on CSA tapering after a maximum of 6 weeks of high dose (5 mg/kg/day) to maintenance dose (2 to 3 mg/kg/day) or a flare after a minimum of 3 months on maintenance dose. Flare is defined as increase in signs or symptoms leading to escalation of therapy, which could be an increase in dose, a switch to a higher potency class of TCS, or the start of another systemic non-steroidal immunosuppressive drug.

Contraindications to CSA include:
i. medical contraindications (e.g. uncontrolled hypertension on medication) or hypersensitivity to CSA active substance or excipients;
ii. use of prohibited concomitant medications (e.g. statins, digoxin, macrolide, antibiotics, barbiturates, anti-seizure, non-steroidal anti-inflammatory drugs, diuretics, angiotensin-converting-enzyme inhibitors, St John's Wort);
iii. increased susceptibility to CSA-induced renal damage (elevated creatinine) and liver damage (elevated function tests), or increased risk of serious infections;
iv. intolerance or unacceptable toxicity (e.g. elevated creatinine, elevated liver function tests, uncontrolled hypertension, paraesthesia, headache, nausea, hypertrichosis), or
v. requirement for CSA at doses >5 mg/kg/day, or duration beyond those specified in the prescribing information (>1 year).

### Treatment of AD

The methods described herein treat AD. Generally, the terms “treat”, “treating”, “treatment”, or the like, mean to alleviate (reduce, minimise, or eliminate) symptoms, or to reduce, minimise or eliminate the causation of symptoms either on a temporary or permanent basis.

### AD-Associated Parameters

Various AD-associated parameters are available to measure the severity of AD and the impact of a drug on AD. These include Investigators Global Assessment (IGA); Eczema Area and Severity Index (EASI); SCORing Atopic Dermatitis (SCORAD); and/or pruritus Numeric Rating Scale (NRS). The methods described herein may improve in an AD-associated parameter in the subject. Alternately, the methods may maintain improvement in an AD-associated parameter in the subject. The AD-associated parameter may be selected from: Investigators Global Assessment (IGA); Eczema Area and Severity Index (EASI); Scoring atopic dermatitis (SCORAD); and/or pruritus Numeric Rating Scale (NRS).

The IGA is an instrument used in clinical trials to rate the severity of the subject's global AD and is based on a 5-point scale ranging from 0 (clear) to 4 (severe) based on the condition of the disease at the time of evaluation.

| | Disease | | IGA morphological |
|---|---|---|---|
| Score | severity | Standard IGA scale | descriptors |
| 0 | Clear | No inflammatory signs of | No erythema and no elevation |
| | | atopic dermatitis | (papulation/infiltration). |
| 1 | Almost clear | Just perceptible erythema, | Barely perceptible erythema |
| | | and just perceptible | and/or minimal lesion elevation |
| | | papulation/infiltration | (papulation/infiltration) that is |
| | | | not widespread. |
| 2 | Mild disease | Mild erythema and mild | Visibly detectable, light pink |
| | | papulation/infiltration | erythema and very slight |
| | | | elevation |
| | | | (papulation/infiltration). |
| 3 | Moderate | Moderate erythema and | Dull red, clearly |
| | disease | moderate | distinguishable erythema and |
| | | papulation/infiltration | clearly perceptible but not |
| | | | extensive elevation |
| | | | (papulation/infiltration). |
| 4 | Severe disease | Severe erythema and severe | Deep/dark red erythema, |
| | | papulation/infiltration | marked and extensive elevation |
| | | | (papulation/infiltration). |

The EASI is a validated measure used in clinical practice and clinical trials to assess the severity and extent of AD (Hanifin et al. “The eczema area and severity index (EASI): assessment of reliability in atopic dermatitis. EASI Evaluator Group. Experimental dermatology” (2001) 10(1): 11-18). SCORAD is one of the most commonly used disease severity scores in clinical trials with AD and in clinical practice (see “European Task Force on Atopic Dermatitis. Severity scoring of atopic dermatitis: the SCORAD index. Consensus report of the European task force on atopic dermatitis” Dermatology (1993) 186(1): 23-31).

Worst Daily Pruritus NRS is established according to FDA and EMA recommendations (see, e.g. FDA “The Food and Drug Administration. Guidance for Industry. Patient-Reported Outcome Measures: Use in Medical Product Development to Support Labeling Claims. 2009”, EMA “Reflection paper on the regulatory guidance for the use of health-related quality of life (HRQoL) measures in the evaluation of medicinal products. EMEA/CHMP/EWP 139391/2004. 2005, and Yosipovitch et al., 2019, “Peak Pruritus Numerical Rating Scale: psychometric validation and responder definition for assessing itch in moderate-to-severe atopic dermatitis”, British Journal of Dermatology (2019) 181, pp. 761-769). For pruritus NRS, a subject assesses their worst itch severity over the past 24 hours using an 11 point NRS (“Worst Daily Pruritus NRS”) from 0 (no itch) to 10 (worst itch imaginable). An average (i.e. mean) Worst Daily Pruritus NRS may be calculated over a certain time period by assessing Worst Daily Pruritus NRS each day and calculating the mean score over that time period. For example, Worst Daily Pruritus may be assessed each day for a one-week, two-week, three-week or four-week period to determine the average (i.e. mean) Worst Daily Pruritus NRS. A Worst Daily Pruritus NRS of <3 represents mild prutius, i.e. only mild itching, with a score of 0 representing no itch.

For each AD-associated parameter, the improvement or maintained improvement is measured relative to baseline. An improvement in this context can be a reduction in IGA score, a reduction in EASI score, a reduction in SCORAD score (where >50 severe, 25-50 is moderate, <25 is mild), a reduction in pruritus NRS score (e.g. Worst Daily Pruritus NRS), where each score is compared to baseline.

The baseline is an initial measurement of an AD-associated parameter or patient-related outcome (or any other parameter) that is taken before initiation of treatment by the method described herein, i.e. a measurement taken before the “baseline dose” (defined elsewhere).

An Investigator's Global Assessment 0 or 1 (IGA 0/1; clear or almost clear skin) and/or ≥75% improvement of Eczema Area and Severity Index (EASI-75) are the regulatory primary efficacy endpoints in Phase 3 clinical trials in AD. Thus, the methods described herein may preferably achieve or maintain an Investigator's Global Assessment (IGA) score of 0 or 1 and/or ≥75% improvement of Eczema Area and Severity Index (EASI-75) over baseline. In some embodiments, the methods may achieve or maintain a ≥50% improvement of Eczema Area and Severity Index (EASI-50) over baseline.

In some embodiments, the method described herein may achieve: (a) ≥50% improvement of Eczema Area and Severity Index (EASI-50); (b) ≥75% improvement of Eczema Area and Severity Index (EASI-75); (c) an IGA score of 0 or 1; (d) ≥2 point reduction of IGA score; (e) a pruritus NRS (e.g. a Worst Daily Pruritus NRS) of <3; and/or (f) ≥3 point reduction in pruritus NRS (e.g. Worst Daily Pruritus NRS).

One or more prior doses of the anti-IL13 antibody, or IL-13-binding fragment thereof, are administered to the subject from 12 days to 16 days (e.g. 14 days) after the immediately preceding dose until the subject achieves an IGA score of 0 or 1 and a Worst Daily Pruritus NRS of <3.

In some embodiments, the one or more prior doses are administered to the subject from 12 days to 16 days (e.g. 14 days) after the immediately preceding dose until the subject achieves an IGA score of 0 or 1 and a Worst Daily Pruritus NRS of <3 (i.e. 0-2) for at least one day, at least two consecutive days, at least three consecutive days, at least four consecutive days, at least five consecutive days, at least six consecutive days, at least one week, at least two consecutive weeks, at least three consecutive weeks or at least four consecutive weeks. In some embodiments, the Worst Daily Pruritus NRS may be assessed daily for one day, at least two consecutive days, at least three consecutive days, at least four consecutive days, at least five consecutive days, at least six consecutive days, at least one week, at least two consecutive weeks, at least three consecutive weeks or at least four consecutive weeks. Preferably, the one or more prior doses are administered to the subject from 12 days to 16 days (e.g. 14 days) after the immediately preceding dose until the subject achieves an IGA score of 0 or 1 and a Worst Daily Pruritus NRS of <3 (i.e. 0-2) for at least four consecutive weeks.

In some embodiments, the one or more prior doses are administered to the subject from 12 days to 16 days (e.g. 14 days) after the immediately preceding dose until the subject achieves a mean IGA score of 0 or 1 and a mean Worst Daily Pruritus NRS of <3 (i.e. 0-2) over a one week, two week, three week or four week period. In some embodiments, the Worst Daily Pruritus may be assessed daily for one day, at least two consecutive days, at least three consecutive days, at least four consecutive days, at least five consecutive days, at least six consecutive days, for at least one week, at least two consecutive weeks, at least three consecutive weeks or at least four consecutive weeks. Preferably, the one or more prior doses are administered to the subject from 12 days to 16 days (e.g. 14 days) after the immediately preceding dose until the subject achieves an IGA score of 0 or 1 and a Worst Daily Pruritus NRS of <3 (i.e. 0-2) for at least four consecutive weeks, or until the subject achieves a mean IGA score of 0 or 1 and a mean Worst Daily Pruritus NRS of <3 (i.e. 0-2) over a four-week time period.

In some embodiments, the one or more prior doses are administered to the subject from 12 days to 16 days (e.g. 14 days) after the immediately preceding dose until the subject achieves clear skin (i.e. an IGA score of 0) and no itch (i.e. a Worst Daily Pruritus NRS of 0). In some embodiments, the one or more prior doses are administered to the subject from 12 days to 16 days (e.g. 14 days) after the immediately preceding dose until the subject achieves clear skin (i.e. a mean IGA score of 0) and no itch (i.e. a mean Worst Daily Pruritus NRS of 0) over a one week, two week, three week or four week period. In such embodiments, Worst daily Pruritus NRS may be assessed daily. In some embodiments, the one or more prior doses are administered to the subject from 12 days to 16 days (e.g. 14 days) after the immediately preceding dose until the subject achieves clear skin (i.e. an IGA score of 0) and no itch (i.e. a Worst Daily Pruritus NRS of 0) for at least one day, two consecutive days, at least three consecutive days, at least four consecutive days, at least five consecutive days, at least six consecutive days, at least one week, at least two consecutive weeks, at least three consecutive weeks or at least four consecutive weeks. In such embodiments, the Worst daily Pruritus NRS may be assessed daily.

In some embodiments, the subject may achieve an IGA score of 0 or 1 and a Worst Daily Pruritus NRS of <3 (i.e. 0-2) after administering the one or more prior doses(s) of an anti-IL-13 antibody, or an IL-13-binding fragment thereof, to the subject from 12 days to 16 days (e.g. 14 days) after the immediately preceding dose for at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, at least 16 weeks, at least 17 weeks, at least 18 weeks, at least 19 weeks, at least 20 weeks, at least 21 weeks, at least 22 weeks, at least 23 weeks, at least 24 weeks, at least 25 weeks, at least 26 weeks, at least 27 weeks, at least 28 weeks, at least 29 weeks, at least 30 weeks, at least 31 weeks or at least 32 weeks. Preferably, the method may comprise administering the one or more prior doses(s) of an anti-IL-13 antibody, or an IL-13-binding fragment thereof, to the subject for from about 12 to 16 weeks. In some embodiments, the one or more prior doses(s) of an anti-IL-13 antibody, or an IL-13-binding fragment thereof, are administered to the subject for about 12 weeks, about 13 weeks, about 14 weeks, about 15 weeks or about 16 weeks (e.g. about 12 weeks or about 16 weeks).

In some embodiments, the subject may achieve an IGA score of 0 or 1 and a Worst Daily Pruritus NRS of <3 (i.e. 0-2) after administering the one or more prior doses(s) of an anti-IL-13 antibody, or an IL-13-binding fragment thereof, to the subject from 12 days to 16 days (e.g. 14 days) after the immediately preceding dose for from about 12 to 16 weeks. In some embodiments, the subject may achieve an IGA score of 0 or 1 and a Worst Daily Pruritus NRS of <3 (i.e. 0-2) after administering the one or more prior doses(s) of an anti-IL-13 antibody, or an IL-13-binding fragment thereof, to the subject from 12 days to 16 days (e.g. 14 days) after the immediately preceding dose for about 12 weeks, about 13 weeks, about 14 weeks, about 15 weeks or about 16 weeks (e.g. about 12 weeks or about 16 weeks).

In some embodiments, the subject may achieve an IGA score of 0 or 1 and a Worst Daily Pruritus NRS of <3 (i.e. 0-2) for at least one day, at least two consecutive days, at least three consecutive days, at least four consecutive days, at least five consecutive days, at least six consecutive days, at least one week, at least two consecutive weeks, at least three consecutive weeks or at least four consecutive weeks after administering the one or more prior doses(s) of an anti-IL-13 antibody, or an IL-13-binding fragment thereof, to the subject from 12 days to 16 days (e.g. 14 days) after the immediately preceding dose for at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, at least 16 weeks, at least 17 weeks, at least 18 weeks, at least 19 weeks, at least 20 weeks, at least 21 weeks, at least 22 weeks, at least 23 weeks, at least 24 weeks, at least 25 weeks, at least 26 weeks, at least 27 weeks, at least 28 weeks, at least 29 weeks, at least 30 weeks, at least 31 weeks and at least 32 weeks.

In some embodiments, the subject may achieve an IGA score of 0 or 1 and a Worst Daily Pruritus NRS of <3 (i.e. 0-2) for at least one day, at least two consecutive days, at least three consecutive days, at least four consecutive days, at least five consecutive days, at least six consecutive days, at least one week, at least two consecutive weeks, at least three consecutive weeks or at least four consecutive weeks after administering the one or more prior doses(s) of an anti-IL-13 antibody, or an IL-13-binding fragment thereof, to the subject from 12 days to 16 days (e.g. 14 days) after the immediately preceding dose for from 12 to 16 weeks.

In some embodiments, the subject may achieve an IGA score of 0 or 1 and a Worst Daily Pruritus NRS of <3 (i.e. 0-2) for at least one day, at least two consecutive days, at least three consecutive days, at least four consecutive days, at least five consecutive days, at least six consecutive days, at least one week, at least two consecutive weeks, at least three consecutive weeks or at least four consecutive weeks after administering the one or more prior doses(s) of an anti-IL-13 antibody, or an IL-13-binding fragment thereof, to the subject from 12 days to 16 days (e.g. 14 days) after the immediately preceding dose for about 12 weeks, about 13 weeks, about 14 weeks, about 15 weeks or about 16 weeks (e.g. about 12 weeks or about 16 weeks).

In some embodiments, the subject may achieve a mean IGA score of 0 or 1 and a mean Worst Daily Pruritus NRS of <3 (i.e. 0-2) over a one week, two week, three week or four week period after administering the one or more prior doses(s) of an anti-IL-13 antibody, or an IL-13-binding fragment thereof, to the subject from 12 days to 16 days (e.g. 14 days) after the immediately preceding dose for from about 12 to about 16 weeks.

In some embodiments, the subject may achieve a mean IGA score of 0 or 1 and a mean Worst Daily Pruritus NRS of <3 (i.e. 0-2) over a one week, two week, three week or four week period after administering the one or more prior doses(s) of an anti-IL-13 antibody, or an IL-13-binding fragment thereof, to the subject from 12 days to 16 days (e.g. 14 days) after the immediately preceding dose for about 12 weeks, about 13 weeks, about 14 weeks, about 15 weeks or about 16 weeks (e.g. about 12 weeks or about 16 weeks).

In some embodiments, the subject may achieve clear skin (i.e. an IGA score of 0) and no itch (i.e. a mean Worst Daily Pruritus NRS of 0) over a one week, two week, three week or four week period after administering the one or more prior doses(s) of an anti-IL-13 antibody, or an IL-13-binding fragment thereof, to the subject from 12 days to 16 days (e.g. 14 days) after the immediately preceding dose for from about 12 to about 16 weeks.

In some embodiments, the subject may achieve clear skin (i.e. an IGA score of 0) and no itch (i.e. a mean Worst Daily Pruritus NRS of 0) over a one week, two week, three week or four week period after administering the one or more prior doses(s) of an anti-IL-13 antibody, or an IL-13-binding fragment thereof, to the subject from 12 days to 16 days (e.g. 14 days) after the immediately preceding dose for about 12 weeks, about 13 weeks, about 14 weeks, about 15 weeks or about 16 weeks (e.g. about 12 weeks or about 16 weeks).

In some embodiments, the subject may achieve clear or almost clear skin (i.e. an IGA score of 0 or 1) and mild itch (i.e. a mean Worst Daily Pruritus NRS of 1 or 2) over a one week, two week, three week or four week period after administering the one or more prior doses(s) of an anti-IL-13 antibody, or an IL-13-binding fragment thereof, to the subject from 12 days to 16 days (e.g. 14 days) after the immediately preceding dose for from about 12 to about 16 weeks.

In some embodiments, the subject may achieve clear or almost clear skin (i.e. an IGA score of 0 or 1) and mild itch (i.e. a mean Worst Daily Pruritus NRS of 1 or 2) over a one week, two week, three week or four week period after administering the one or more prior doses(s) of an anti-IL-13 antibody, or an IL-13-binding fragment thereof, to the subject from 12 days to 16 days (e.g. 14 days) after the immediately preceding dose for about 12 weeks, about 13 weeks, about 14 weeks, about 15 weeks or about 16 weeks (e.g. about 12 weeks or about 16 weeks).

Once the subject has achieved an IGA score of 0 or 1 and a worst daily pruritus NRS of <3 (i.e. 0, 1 or 2) for the specified time period according to any of the embodiments above, the subject is moved to a maintenance phase of treatment, such that the anti-IL-13 antibody, or IL-13-binding fragment thereof, is administered to the subject at a reduced dosing frequency. In this phase, the method comprises administering a first dose of the anti-IL-13 antibody, or IL-13-binding fragment thereof, to the subject; and administering one or more secondary dose(s) of the anti-IL-13 antibody, or IL-13-binding fragment thereof, to the subject, wherein each secondary dose is administered to the subject from 25 days to 31 days, e.g. 28 days, after the immediately preceding dose.

### Treatment of Pruritus

In some aspects, the methods described herein treat pruritus (i.e. itching).

Treatment of pruritus means a reduction of Worst Daily Pruritus Numerical Rating Score (NRS) compared to baseline, e.g. before treatment. In some embodiments, treatment of pruritus is characterised by a ≥1-point reduction, a ≥2-point reduction, a ≥3-point reduction, or a ≥4-point reduction in Worst Daily Pruritus NRS from baseline e.g. before treatment. Preferably, treatment of pruritus is characterised by the subject achieving a Worst Daily Pruritus NRS of <3 (i.e. 0-2), which represents “no itch” or “mild itch”.

Worst Daily Pruritus NRS is assessed as described above under “AD-associated parameters”.

In some embodiments, the one or more prior doses are administered to the subject from 12 days to 16 days (e.g. 14 days) after the immediately preceding dose until the subject achieves a Worst Daily Pruritus NRS of <3 (i.e. 0-2) for at least one day, at least two consecutive days, at least three consecutive days, at least four consecutive days, at least five consecutive days, at least six consecutive days, at least one week, at least two consecutive weeks, at least three consecutive weeks or at least four consecutive weeks. In such embodiments, the Worst Daily Pruritus may be assessed daily for at least one day, at least two consecutive days, at least three consecutive days, at least four consecutive days, at least five consecutive days, at least six consecutive days, at least one week, at least two consecutive weeks, at least three consecutive weeks or at least four consecutive weeks. Preferably, the one or more prior doses are administered to the subject from 12 days to 16 days (e.g. 14 days) after the immediately preceding dose until the subject achieves a Worst Daily Pruritus NRS of <3 (i.e. 0-2) for at least four consecutive weeks.

In some embodiments, the one or more prior doses are administered to the subject from 12 days to 16 days (e.g. 14 days) after the immediately preceding dose until the subject achieves a mean Worst Daily Pruritus NRS of <3 (i.e. 0-2) over a one week, two week, three week or four week period. In such embodiments, the Worst Daily Pruritus may be assessed daily for at least two consecutive days, at least three consecutive days, at least four consecutive days, at least five consecutive days, at least six consecutive days, for at least one week, at least two consecutive weeks, at least three consecutive weeks or at least four consecutive weeks. Preferably, the one or more prior doses are administered to the subject from 12 days to 16 days (e.g. 14 days) after the immediately preceding dose until the subject achieves a Worst Daily Pruritus NRS of <3 (i.e. 0-2) for at least four consecutive weeks, or until the subject achieves a mean Worst Daily Pruritus NRS of <3 (i.e. 0-2) over a four-week time period.

In some embodiments, the one or more prior doses are administered to the subject from 12 days to 16 days (e.g. 14 days) after the immediately preceding dose until the subject achieves no itch (i.e. a Worst Daily Pruritus NRS of 0). In some embodiments, the one or more prior doses are administered to the subject from 12 days to 16 days (e.g. 14 days) after the immediately preceding dose until the subject achieves no itch (i.e. a mean Worst Daily Pruritus NRS of 0) over a one week, two week, three week or four week period. In such embodiments, the Worst daily Pruritus NRS may be assessed daily. In some embodiments, the one or more prior doses are administered to the subject from 12 days to 16 days (e.g. 14 days) after the immediately preceding dose until the subject achieves no itch (i.e. a Worst Daily Pruritus NRS of 0) for at least one day, at least two consecutive days, at least three consecutive days, at least four consecutive days, at least five consecutive days, at least six consecutive days, at least one week, at least two consecutive weeks, at least three consecutive weeks or at least four consecutive weeks. In such embodiments, the Worst daily Pruritus NRS may be assessed daily.

In some embodiments, the subject may achieve a Worst Daily Pruritus NRS of <3 (i.e. 0-2) after administering the one or more prior doses(s) of an anti-IL-13 antibody, or an IL-13-binding fragment thereof, to the subject from 12 days to 16 days (e.g. 14 days) after the immediately preceding dose for at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, at least 16 weeks, at least 17 weeks, at least 18 weeks, at least 19 weeks, at least 20 weeks, at least 21 weeks, at least 22 weeks, at least 23 weeks, at least 24 weeks, at least 25 weeks, at least 26 weeks, at least 27 weeks, at least 28 weeks, at least 29 weeks, at least 30 weeks, at least 31 weeks or at least 32 weeks. Preferably, the method may comprise administering the one or more prior doses(s) of an anti-IL-13 antibody, or an IL-13-binding fragment thereof, to the subject for from about 12 to about 16 weeks. In some embodiments, the one or more prior doses(s) of an anti-IL-13 antibody, or an IL-13-binding fragment thereof, are administered to the subject for about 12 weeks, about 13 weeks, about 14 weeks, about 15 weeks or about 16 weeks (e.g. 12 weeks or 16 weeks).

In some embodiments, the subject may achieve a Worst Daily Pruritus NRS of <3 (i.e. 0-2) after administering the one or more prior doses(s) of an anti-IL-13 antibody, or an IL-13-binding fragment thereof, to the subject from 12 days to 16 days (e.g. 14 days) after the immediately preceding dose for from about 12 to about 16 weeks. In some embodiments, the subject may achieve a Worst Daily Pruritus NRS of <3 (i.e. 0-2) after administering the one or more prior doses(s) of an anti-IL-13 antibody, or an IL-13-binding fragment thereof, to the subject from 12 days to 16 days (e.g. 14 days) after the immediately preceding dose for about 12 weeks, about 13 weeks, about 14 weeks, about 15 weeks or about 16 weeks (e.g. about 12 weeks or about 16 weeks).

In some embodiments, the subject may achieve a Worst Daily Pruritus NRS of <3 (i.e. 0-2) for at least one day, at least two consecutive days, at least three consecutive days, at least four consecutive days, at least five consecutive days, at least six consecutive days, at least one week, at least two consecutive weeks, at least three consecutive weeks or at least four consecutive weeks after administering the one or more prior doses(s) of an anti-IL-13 antibody, or an IL-13-binding fragment thereof, to the subject from 12 days to 16 days (e.g. 14 days) after the immediately preceding dose for at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, at least 16 weeks, at least 17 weeks, at least 18 weeks, at least 19 weeks, at least 20 weeks, at least 21 weeks, at least 22 weeks, at least 23 weeks, at least 24 weeks, at least 25 weeks, at least 26 weeks, at least 27 weeks, at least 28 weeks, at least 29 weeks, at least 30 weeks, at least 31 weeks and at least 32 weeks.

In some embodiments, the subject may achieve a Worst Daily Pruritus NRS of <3 (i.e. 0-2) for at least one day, at least two consecutive days, at least three consecutive days, at least four consecutive days, at least five consecutive days, at least six consecutive days, at least one week, at least two consecutive weeks, at least three consecutive weeks or at least four consecutive weeks after administering the one or more prior doses(s) of an anti-IL-13 antibody, or an IL-13-binding fragment thereof, to the subject from 12 days to 16 days (e.g. 14 days) after the immediately preceding dose for from about 12 to about 16 weeks.

In some embodiments, the subject may achieve a Worst Daily Pruritus NRS of <3 (i.e. 0-2) for at least one day, at least two consecutive days, at least three consecutive days, at least four consecutive days, at least five consecutive days, at least six consecutive days, at least one week, at least two consecutive weeks, at least three consecutive weeks or at least four consecutive weeks after administering the one or more prior doses(s) of an anti-IL-13 antibody, or an IL-13-binding fragment thereof, to the subject from 12 days to 16 days (e.g. 14 days) after the immediately preceding dose for about 12 weeks, about 13 weeks, about 14 weeks, about 15 weeks or about 16 weeks (e.g. about 12 weeks or about 16 weeks).

In some embodiments, the subject may achieve a mean Worst Daily Pruritus NRS of <3 (i.e. 0-2) over a one week, two week, three week or four week period after administering the one or more prior doses(s) of an anti-IL-13 antibody, or an IL-13-binding fragment thereof, to the subject from 12 days to 16 days (e.g. 14 days) after the immediately preceding dose for from about 12 to about 16 weeks.

In some embodiments, the subject may achieve a mean Worst Daily Pruritus NRS of <3 (i.e. 0-2) over a one week, two week, three week or four week period after administering the one or more prior doses(s) of an anti-IL-13 antibody, or an IL-13-binding fragment thereof, to the subject from 12 days to 16 days (e.g. 14 days) after the immediately preceding dose for about 12 weeks, about 13 weeks, about 14 weeks, about 15 weeks or about 16 weeks (e.g. about 12 weeks or about 16 weeks).

In some embodiments, the subject may achieve no itch (i.e. a mean Worst Daily Pruritus NRS of 0) over a one week, two week, three week or four week period after administering the one or more prior doses(s) of an anti-IL-13 antibody, or an IL-13-binding fragment thereof, to the subject from 12 days to 16 days (e.g. 14 days) after the immediately preceding dose for from about 12 to about 16 weeks.

In some embodiments, the subject may achieve no itch (i.e. a mean Worst Daily Pruritus NRS of 0) over a one week, two week, three week or four week period after administering the one or more prior doses(s) of an anti-IL-13 antibody, or an IL-13-binding fragment thereof, to the subject from 12 days to 16 days (e.g. 14 days) after the immediately preceding dose for about 12 weeks, about 13 weeks, about 14 weeks, about 15 weeks or about 16 weeks (e.g. about 12 weeks or about 16 weeks).

In some embodiments, the subject may achieve mild itch (i.e. a mean Worst Daily Pruritus NRS of 1 or 2) over a one week, two week, three week or four week period after administering the one or more prior doses(s) of an anti-IL-13 antibody, or an IL-13-binding fragment thereof, to the subject from 12 days to 16 days (e.g. 14 days) after the immediately preceding dose for from about 12 to about 16 weeks.

In some embodiments, the subject may achieve mild itch (i.e. a mean Worst Daily Pruritus NRS of 1 or 2) over a one week, two week, three week or four week period after administering the one or more prior doses(s) of an anti-IL-13 antibody, or an IL-13-binding fragment thereof, to the subject from 12 days to 16 days (e.g. 14 days) after the immediately preceding dose for about 12 weeks, about 13 weeks, about 14 weeks, about 15 weeks or about 16 weeks (e.g. about 12 weeks or about 16 weeks).

Once the subject has achieved a worst daily pruritus NRS of <3 (i.e. 0, 1 or 2) for the specified time period, according to any one of the embodiments above, the subject is moved to a maintenance phase of treatment, such that the anti-IL-13 antibody, or IL-13-binding fragment thereof, is administered to the subject at a reduced dosing frequency. In this phase, the method comprises administering a first dose of the anti-IL-13 antibody, or IL-13-binding fragment thereof, to the subject; and administering one or more secondary dose(s) of the anti-IL-13 antibody, or IL-13-binding fragment thereof, to the subject, wherein each secondary dose is administered to the subject from 25 days to 31 days, e.g. 28 days, after the immediately preceding dose.

### Anti-IL-13 Antibodies and IL-13-Binding Fragments Thereof

An anti-IL-13 antibody is an antibody that specifically binds to and neutralizes IL-13, e.g. human IL-13.

Methods for identifying, isolating and testing (e.g. binding and neutralisation) of antibodies and fragments thereof are well-known in the art. See WO 2005/007699, which teaches the identification and characterisation of various anti-IL13 antibodies and fragments and provides suitable methods for doing so.

Herein, the term “specifically binds” means that an antibody or an antigen-binding fragment thereof forms a complex with an antigen that is relatively stable under physiological conditions. Methods for determining whether an antibody specifically binds to an antigen are well known in the art and include, for example, equilibrium dialysis, surface plasmon resonance (e.g. using a BIAcore 200 Biosensor (BIAcore AB), and the like. For example, an anti-IL-13 antibody or IL-13-binding fragment thereof that “specifically binds” IL-13 may bind IL-13 with a K_{D} of less than about 1000 nM, less than about 500 nM, less than about 100 nM, less than about 50 nM, less than about 20 nM, less than about 10 nM, less than about 5 nM, less than about 1 nM, less than about 0.5 nM, less than about 0.25 nM, less than about 0.1 nM or less than about 0.05 nM, as measured by surface plasmon resonance at 25° C. The exemplified antibody tralokinumab binds human bound human IL-13 with a K_{D} of 178 pM, as measured by surface plasmon resonance (see WO 2005/007699 for detailed methods). Accordingly, in a preferred embodiment, the anti-IL-13 antibody has a K_{D} of less than about 200 pM, as measured by surface plasmon resonance at 37° C. or 25° C. Although an IL-13 antibody or IL-13-binding fragment thereof specifically binds human IL-13, it may have cross-reactivity to other antigens, such as IL-13 from other (non-human) species.

The anti-IL-13 antibody, or IL-13-binding fragment thereof, may inhibit or neutralise at least one downstream activity of IL-13. Methods for measuring neutralisation activity are well known in the art. Neutralisation potency may be assessed using any suitable assay known in the art, such as a TF-1 factor dependent cell proliferation assay, an HDLM-2 cell proliferation assay, a human umbilical vein endothelial cell (HUVEC) assay, a B9 cell proliferation assay, a human IL-13 dependent peripheral blood mononuclear cell (PBMC) CD23 expression assay or IgE release from human B cells, as described in WO 2005/007699, for example.

In one embodiment, neutralisation activity can be measured in an IL-13 dependent TF-1 cell proliferation assay relative to a control antibody that is not directed to IL-13, as described in WO 2005/007699. In this assay, inhibition of IL-13 dependent proliferation is determined by measuring the reduction in incorporation of tritiated thymidine into the newly synthesized DNA of dividing cells. Briefly, commercial TF-1 cells are maintained according to supplied protocols. Assay media comprises RPMI-1640 with GLUTAMAX I (Invitrogen) containing 5% FBS and 1% sodium pyruvate. Prior to each assay, TF-1 cells are pelleted by centrifugation at 300×g for 5 minutes, the media removed by aspiration and the cells resuspended in assay media. This process is repeated twice with cells resuspended at a final concentration of 10⁵ cells/mL in assay media. Test solutions of antibody (in triplicate) are diluted to the desired concentration in assay media. An antibody that is not directed at IL-13 is used as a negative control. Recombinant bacterially derived human or murine IL-13 is added to a final concentration of 50 ng/mL when mixed with the appropriate test antibody in a total volume of 100 μL/well in a 96 well assay plate. The concentration of IL-13 used in the assay is selected as the dose that at final assay concentration gives approximately 80% of the maximal proliferative response. All samples are incubated for 30 minutes at room temperature. 100 μL of resuspended cells are then added to each assay point to give a total assay volume of 200 μL/well. Assay plates are incubated for 72 hours at 37° C. under 5% CO₂. 25 μL of tritiated thymidine (10 μCi/mL) is then added to each assay point and assay plates are returned to the incubator for a further 4 hours. Cells are harvested on glass fibre filter plates (Perkin Elmer) using a cell harvester. Thymidine incorporation is determined using a Packard TopCount microplate liquid scintillation counter.

The term “antibody”, as used herein, includes immunoglobulin molecules comprising four polypeptide chains, two heavy (H) chains and two light (L) chains inter-connected by disulfide bonds, as well as multimers thereof (e.g. IgM). In a typical antibody, each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. In some cases, the FRs of the anti-IL-13 antibody (or IL-13-binding fragment or derivative thereof) may be identical to the human germline sequences, or may be naturally or artificially modified.

The heavy chain constant region of the antibodies may be from any types of constant region, such as IgG, IgM, IgD, IgA, and IgE. Generally, the antibody is an IgG (e.g. isotype IgG1, IgG2, IgG3 or IgG4). Preferably, the antibody is an IgG4, as exemplified herein.

The antibody may be a mouse, human, primate, humanized or chimeric antibody. The antibody may be polyclonal or monoclonal. For therapeutic applications, monoclonal and human (or humanized) antibodies are preferred. In a particularly preferred embodiment, the antibody is human or humanized, and monoclonal.

The antibody can be a multispecific (e.g. bispecific) antibody. A multispecific antibody or antigen-binding fragment of an antibody will typically comprise at least two different variable domains, wherein each variable domain is capable of specifically binding to a separate antigen or to a different epitope on the same antigen. Any multispecific antibody format may be adapted for use in the context of an antibody or antigen binding fragment of an antibody as described herein using routine techniques available in the art. For example, the methods that use of bispecific antibodies, wherein one arm of an immunoglobulin is specific for IL-13, and the other arm of the immunoglobulin is specific for a second therapeutic target or is conjugated to a therapeutic moiety.

An IL-13-binding fragment of an anti-IL-13 antibody may be any naturally occurring, enzymatically obtainable, synthetic, or genetically engineered polypeptide. Such fragments may be derived, e.g. from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g. commercial sources, DNA libraries (including, e.g. phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of IL-13-binding fragments include: Fab, Fab′, F(ab′)2, Fd, Fv, single-chain Fv (scFv), disulphide-linked Fvs, dAb fragments, and other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains.

An IL-13-binding fragment of an anti-IL-13-binding antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

The anti-IL-13 antibody, or an IL-13-binding fragment thereof, may comprise: a heavy chain complementarity determining region 1 (HCDR1) comprising an amino acid sequence of SEQ ID NO: 1; a heavy chain complementarity determining region 2 (HCDR2) comprising an amino acid sequence of SEQ ID NO:2; a heavy chain complementarity determining region 3 (HCDR3) comprising an amino acid sequence of SEQ ID NO:3; a light chain complementarity determining region 1 (LCDR1) comprising an amino acid sequence of SEQ ID NO:4; a light chain complementarity determining region 2 (LCDR2) comprising an amino acid sequence of SEQ ID NO:5; and a light chain complementarity determining region 3 (LCDR3) comprising an amino acid sequence of SEQ ID NO:6. The anti-IL-13 antibody, or an IL-13-binding fragment thereof, may comprise a heavy chain variable region (HCVR) and a light chain variable region (LCVR), wherein: (i) the heavy chain variable region comprises: a heavy chain complementarity determining region 1 (HCDR1) comprising an amino acid sequence of SEQ ID NO:1; a heavy chain complementarity determining region 2 (HCDR2) comprising an amino acid sequence of SEQ ID NO:2; and a heavy chain complementarity determining region 3 (HCDR3) comprising an amino acid sequence of SEQ ID NO:3; and (ii) the light chain variable region comprises: a light chain complementarity determining region 1 (LCDR1) comprising an amino acid sequence of SEQ ID NO:4; a light chain complementarity determining region 2 (LCDR2) comprising an amino acid sequence of SEQ ID NO:5; and a light chain complementarity determining region 3 (LCDR3) comprising an amino acid sequence of SEQ ID NO:6. In addition, the anti-IL-13 antibody, or an IL-13-binding fragment thereof, may further comprise: (i) an amino acid sequence that is at least 80%, at least 85%, at least 90%, at least 95%, at least 96%, at least 97%, at least 98%, or at least 99% identical to a heavy chain variable region sequence of SEQ ID NO: 8; and/or (ii) an amino acid sequence that is 80%, at least 85%, at least 90%, at least 95%, at least 96%, at least 97%, at least 98%, or at least 99% identical to a light chain variable region sequence of SEQ ID NO: 10. The anti-IL-13 antibody, or an IL-13-binding fragment thereof, may comprise a heavy chain variable region sequence of SEQ ID NO: 8 and a light chain variable region sequence of SEQ ID NO: 10.

The anti-IL-13 antibody, or the IL-13-binding fragment thereof, may comprise: (i) an amino acid sequence that is at least 80%, at least 85%, at least 90%, at least 95%, at least 96%, at least 97%, at least 98%, or at least 99% identical to the heavy chain sequence of SEQ ID NO: 11; and/or (ii) an amino acid sequence that is at least 80%, at least 85%, at least 90%, at least 95%, at least 96%, at least 97%, at least 98%, or at least 99% identical to the light chain sequence of SEQ ID NO: 12. In some cases, the anti-IL-13 antibody, or an IL-13-binding fragment or IL-13-binding derivative thereof, comprises a heavy chain of SEQ TD NO: 11 and a light chain sequence of SEQ ID NO: 12.

One such antibody that can be used in the methods described herein is the anti-IL-13 antibody, tralokinumab (as described in the “International Nonproprietary Names for Pharmaceutical Substances (INN)” list 102 (WHO Drug Information (2009) 23(4): pp 348)). Tralokinumab is a fully human IgG4-lambda antibody, which specifically binds and neutralises human IL-13.

TABLE 1

| | | |
|---|---|---|
| SEQ ID | | |
| number | Name | Sequence |

Tralokinumab

| | | |
|---|---|---|
| SEQ ID NO: 1 | HCDR1 | NYGLS |
| SEQ ID NO: 2 | HCDR2 | WISANNGDTNYGQEFQG |
| SEQ ID NO: 3 | HCDR3 | DSSSSWARWFFDL |
| SEQ ID NO: 4 | LCDR1 | GGNIIGSKL VH |
| SEQ ID NO: 5 | LCDR2 | DDGDRPS |
| SEQ ID NO: 6 | LCDR3 | QVWDTGSDPVV |
| SEQ ID NO: 7 | cDNA heavy | caggtccagctggtgcagtctggggctgaggtgaagaagcctgggg |
| | chain variable | cctcagtgaaggtctcctgcaaggcttctggttacacctttacaaattat |
| | domain | ggtctcagctgggtgcgacaggcccctggacaagggcttgagtggat |
| | | gggatggatcagcgctaataatggcgacacaaattatggacaggaatt |
| | | ccagggcagagtcaccatgaccacagatacatccacgagcacagcc |
| | | tacatggagttgaggagcctgagatctgacgacacggccgtttattact |
| | | gtgcgagagactccagcagcagctgggcccgctggtttttcgatctct |
| | | ggggccgggggacactggtcaccgtctcctca |
| SEQ ID NO: 8 | polypeptide | QVQLVQSGAEVKKPGASVKVSCKASGYTFT |
| | sequence heavy | NYGLSWVRQAPGQGLEWMGWISANNGDTN |
| | chain variable | YGQEFQGRVTMTTDTSTSTAYMELRSLRSD |
| | region | DTAVYYCARDSSSSWARWFFDLWGRGTLV |
| | | TVSS |
| SEQ ID NO: 9 | CDNA light | tcctatgtgctgactcagccaccctcggtgtcagtggccccaggaaag |
| | chain variable | acggccaggattacctgtgggggaaacatcattggaagtaaacttgta |
| | domain | cactggtaccagcagaagccaggccaggcccctgtgctggtcatcta |
| | | tgatgatggcgaccggccctcagggatccctgagcgattctctggctc |
| | | caactctgggaacacggccaccctgaccatcagcagggtcgaggcc |
| | | ggggatgaggccgactattattgtcaggtgtgggatactggtagtgat |
| | | cccgtggtattcggcggagggaccaagctgaccgtcctaggt |
| SEQ ID NO: 10 | polypeptide | SYVLTQPPSVSVAPGKTARITCGGNIIGSKLV |
| | sequence light | HWYQQKPGQAPVLVIYDDGDRPSGIPERFSG |
| | chain variable | SNSGNTATLTISRVEAGDEADYYCQVWDTG |
| | region | SDPVVFGGGTKLTVL |
| SEQ ID NO: 11 | Heavy chain | QVQLVQSGAEVKKPGASVKVSCKASGYTFT |
| | | NYGLSWVRQAPGQGLEWMGWISANNGDTN |
| | | YGQEFQGRVTMTTDTSTSTAYMELRSLRSD |
| | | DTAVYYCARDSSSSWARWFFDLWGRGTLV |
| | | TVSSASTKGPSVFPLAPCSRSTSESTAALGCL |
| | | VKDYFPEPVTVSWNSGALTSGVHTFPAVLQS |
| | | SGLYSLSSVVTVPSSSLGTKTYTCNVDHKPS |
| | | NTKVDKRVESKYGPPCPSCPAPEFLGGPSVF |
| | | LFPPKPKDTLMISRTPEVTCVVVDVSQEDPE |
| | | VQFNWYVDGVEVHNAKTKPREEQFNSTYR |
| | | VVSVLTVLHQDWLNGKEYKCKVSNKGLPSS |
| | | IEKTISKAKGQPREPQVYTLPPSQEEMTKNQ |
| | | VSLTCLVKGFYPSDIAVEWESNGQPENNYKT |
| | | TPPVLDSDGSFFLYSRLTVDKSRWQEGNVFS |
| | | CSVMHEALHNHYTQKSLSLSLGK |
| SEQ ID NO: 12 | Light chain | SYVLTQPPSVSVAPGKTARITCGGNIIGSKLV |
| | | HWYQQKPGQAPVLVIYDDGDRPSGIPERFSG |
| | | SNSGNTATLTISRVEAGDEADYYCQVWDTG |
| | | SDPVVFGGGTKLTVLGQPKAAPSVTLFPPSS |
| | | EELQANKATLVCLISDFYPGAVTVAWKADS |
| | | SPVKAGVETTTPSKQSNNKYAASSYLSLTPE |
| | | QWKSHRSYSCQVTHEGSTVEKTVAPTECS |

### Dose and Dosing Regimen

The invention relates to a method of treating atopic dermatitis (AD) in a subject in need thereof, wherein the method comprises the steps of:
(a) administering one or more prior dose(s) of the anti-IL-13 antibody, or IL-13-binding fragment thereof, to the subject, wherein each prior dose is administered from 12 days to 16 days after the immediately preceding dose, until the subject achieves an Investigator's Global Assessment (IGA) score of 0 or 1 and a worst Daily Pruritus Numeric Rating Scale (NRS) of <3;
(b) administering a first dose of the anti-IL-13 antibody, or IL-13-binding fragment thereof, to the subject; and
(c) administering one or more secondary dose(s) of the anti-IL-13 antibody, or IL-13-binding fragment thereof, to the subject, wherein each secondary dose is administered to the subject from 25 days to 31 days after the immediately preceding dose.

Optionally, the method comprises the step of administering a loading dose of the anti-IL-13 antibody, or IL-13-binding fragment thereof, to the subject before step (a) of administering one or more prior dose(s) of the of the anti-IL-13 antibody, or IL-13-binding fragment thereof, to the subject.

The term “dose” refers to the amount (mass) of anti-IL-13 antibody, or IL-13-binding fragment thereof, administered to the subject on the particular treatment day. For example, a dose of 300 mg of anti-IL-13 antibody, or IL-13-binding fragment thereof, means that on a treatment day a total of 300 mg of anti-IL-13 antibody, or IL-13-binding fragment thereof, is given to the subject. Typically, a dose is administered in a single administration step (e.g. one injection). However, in some embodiments, one, two, three or more administration steps (e.g. one, two, three or more injections) may be used to provide the subject with the desired dose.

The terms “prior dose”, “first dose”, and “secondary dose” refer to the temporal sequence of administration of the anti-IL-13 antibody, or IL-13-binding fragment thereof. The term “first dose” is a single dose of anti-IL-13 antibody, or IL-13-binding fragment thereof, that is followed by one or more secondary dose(s). The first dose is preceded by one or more prior dose(s). Subsequent to the first dose is one or more secondary dose(s).

The phrase “immediately preceding dose” means, in a sequence of multiple doses, the dose of anti-IL-13 antibody, or IL-13-binding fragment thereof, which is administered to a patient prior to the administration of the very next dose in the sequence, with no intervening doses of the anti-IL-13 antibody, or IL-13-binding fragment thereof.

“Dosing frequency” is the frequency of administering a dose of the anti-IL-13 antibody, or IL-13-binding fragment thereof. Thus, a decrease in dosing frequency means an increase in the time interval between doses. Common terminology used in relation to dosing frequency is QW (once weekly), Q2W (once every 2 weeks), Q3W (every 3 weeks), or Q4W (every 4 weeks).

Each prior dose may be from about 10 mg to about 600 mg of the anti-IL-13 antibody, or IL-13-binding fragment thereof, from about 50 mg to 500 mg, from about 100 mg to about 400 mg, from about 250 mg to about 350 mg or from about 280 mg to about 320 mg of the anti-IL-13 antibody, or IL-13-binding fragment thereof. For example, the prior dose may be about 10 mg, about 25 mg, about 50 mg, about 100 mg, about 150 mg, about 200 mg, about 250 mg, about 300 mg, about 400 mg or about 500 mg. In some cases, the prior dose may be about 600 mg or less, about 500 mg or less, about 400 mg or less, about 300 mg or less, about 200 mg or less, or about 200 mg or less. In some embodiments, the prior dose is about 150 mg of anti-IL-13 antibody, or IL-13 binding fragment thereof. In preferred embodiments, the prior dose is about 300 mg of anti-IL-13 antibody, or IL-13-binding fragment thereof.

Optionally, an initial loading dose of the anti-IL-13 antibody, or IL-13-binding fragment thereof, is administered to the subject before step (a) of administering one or more prior dose(s) of the of the anti-IL-13 antibody, or IL-13-binding fragment thereof, to the subject. This initial loading dose may be a bolus dose which is double the amount of the one or more prior dose(s) following the bolus dose. In some embodiments, the initial loading dose is a 600 mg dose and the one or more prior dose(s) following the 600 mg dose are 300 mg dose(s). In some embodiments, the initial loading dose is a 300 mg dose and the one or more prior dose(s) following the 300 mg dose are 150 mg dose(s).

As such, a bolus dose is given as a first dose of the above mentioned “one or more prior doses” or as the first dose in step (b). The bolus is typically twice the amount of the dose administered with the next administration. For example, a dose of 600 mg is used as a bolus dose when the next dose administered is 300 mg, and a dose of 300 mg is used as a bolus dose when the next dose administered is 150 mg.

In a preferred embodiment of the methods of treating AD described herein, the one or more prior dose(s) the anti-IL-13 antibody (e.g. Tralokinumab), or IL-13-binding fragment thereof, are administered to the subject from 12 days to 16 days (e.g. 14 days) after the immediately preceding dose for about 12 to about 16 weeks (e.g. for about 12 weeks or about 16 weeks), wherein the subject has achieved an IGA score of 0 or 1 and a Worst Daily Pruritus NRS of <3 (i.e. 0-2) for at least one week, at least two consecutive weeks, at least three consecutive weeks or at least four consecutive weeks, or a mean IGA score of 0 or 1 and a mean Worst Daily Pruritus NRS of <3 (i.e. 0-2) over a one week, two week, three week or four week period, and wherein each of the one or more prior dose(s) is about 300 mg. In some embodiments, an initial loading dose (e.g. a bolus dose of about 600 mg) of the anti-IL-13 antibody (e.g. Tralokinumab), or IL-13-binding fragment thereof, is administered to the subject before step (a) of administering one or more prior dose(s) of the of the anti-IL-13 antibody, or IL-13-binding fragment thereof, to the subject.

Once the subject has achieved an IGA score of 0 or 1 and a worst daily pruritus NRS of <3 (i.e. 0, 1 or 2) for the specified time period, the subject is moved to a maintenance phase of treatment, such that the anti-IL-13 antibody (e.g. Tralokinumab), or IL-13-binding fragment thereof, is administered to the subject at a reduced dosing frequency. In this phase, the method comprises administering a first dose of the anti-IL-13 antibody (e.g. Tralokinumab), or IL-13-binding fragment thereof, to the subject; and administering one or more secondary dose(s) of the anti-IL-13 antibody (e.g. Tralokinumab), or IL-13-binding fragment thereof, to the subject, wherein each secondary dose is administered to the subject from 25 days to 31 days, e.g. 28 days, after the immediately preceding dose.

The first dose of the anti-IL-13 antibody (e.g. Tralokinumab), or IL-13-binding fragment thereof, may be from about 10 mg to about 600 mg of the anti-IL-13 antibody (e.g. Tralokinumab), or IL-13-binding fragment thereof, from about 50 mg to 500 mg, from about 100 mg to about 400 mg, from about 250 mg to about 350 mg or from about 280 mg to about 320 mg of the anti-IL-13 antibody (e.g. Tralokinumab), or IL-13-binding fragment thereof. For example, the first dose may be about 10 mg, about 25 mg, about 50 mg, about 100 mg, about 150 mg, about 200 mg, about 250 mg, about 300 mg, about 400 mg or about 500 mg. In some cases, the first dose is 600 mg or less, 500 mg or less, 400 mg or less, 300 mg or less, 200 mg or less, or 200 mg or less. In some embodiments, the first dose is about 150 mg of the anti-IL-13 antibody (e.g. Tralokinumab), or IL-13-binding fragment thereof. In preferred embodiments, the first dose is about 300 mg of the anti-IL-13 antibody (e.g. Tralokinumab), or IL-13-binding fragment thereof.

Each secondary dose of the anti-IL-13 antibody (e.g. Tralokinumab), or IL-13-binding fragment thereof, may be administered to the subject from 18 days to 35 days, from 21 days to 35 days, from 22 days to 34 days, from 24 days to 32 days, from 25 days to 31 days, from 26 days to 30 days, or from 27 days to 29 days after the immediately preceding dose. In preferred embodiments, each secondary dose of the anti-IL-13 antibody (e.g. Tralokinumab), or IL-13-binding fragment thereof, is administered to the subject about 28 days after the immediately preceding dose.

Administering one or more secondary dose(s) of the anti-IL-13 antibody, or IL-13-binding fragment thereof, to the subject may be continued (i.e. by administering more than one secondary dose) for at least 8 weeks, at least 12 weeks, at least 16 weeks, at least 20 weeks, at least 24 weeks, at least 28 weeks, at least 32 weeks, at least 36 weeks, at least 40 weeks, at least 44 weeks, at least 48 weeks or at least 52 weeks or more. Administering one or more secondary dose(s) of the anti-IL-13 antibody, or IL-13-binding fragment thereof, to the subject may be continued (i.e. by administering more than one secondary dose) for around 8 weeks, around 12 weeks, around 16 weeks, around 20 weeks, around 24 weeks, around 28 weeks, around 32 weeks, around 36 weeks, around 40 weeks, around 44 weeks, around 48 weeks, or around 52 weeks or more. Additionally, or alternatively, the step of administering one or more secondary dose(s) of the anti-IL-13 antibody, or IL-13-binding fragment thereof, may be continued until the method provides improvement in an AD-associated parameter and/or patient-related outcome as described herein. Administering the anti-IL-13 antibody, or IL-13-binding fragment thereof, may be continued to maintain improvement in an AD-associated parameter and/or patient-related outcome as described herein. In particular cases, administration of the anti-IL-13 antibody (e.g. Tralokinumab), or IL-13-binding fragment thereof, may be continued until the subject reaches a low disease state. Preferably, the anti-IL-13 antibody, or IL-13-binding fragment thereof, is administered until the subject achieves an Investigator's Global Assessment (IGA) score of 0 or 1 and/or ≥75% improvement of Eczema Area and Severity Index (EASI-75) over baseline in the subject.

Each secondary dose may be from about 10 mg to about 600 mg of the anti-IL-13 antibody (e.g. Tralokinumab), or IL-13-binding fragment thereof, from about 50 mg to 500 mg, from about 100 mg to about 400 mg, from about 250 mg to about 350 mg or from about 280 mg to about 320 mg of the anti-IL-13 antibody (e.g. Tralokinumab), or IL-13-binding fragment thereof. For example, each secondary dose may be about 10 mg, about 25 mg, about 50 mg, about 100 mg, about 150 mg, about 200 mg, about 250 mg, about 300 mg, about 400 mg or about 500 mg. In some cases, each secondary dose is 600 mg or less, 500 mg or less, 400 mg or less, 300 mg or less, 200 mg or less or 200 mg or less. In some embodiments, each secondary dose is about 150 mg of the anti-IL-13 antibody (e.g. Tralokinumab), or IL-13-binding fragment thereof. In preferred embodiments, each secondary dose is about 300 mg of the anti-IL-13 antibody (e.g. Tralokinumab), or IL-13-binding fragment thereof. Typically, the first dose and one or more secondary dose(s) are the same amount (i.e. in milligrams) of anti-IL-13 antibody (e.g. Tralokinumab), or IL-13-binding fragment thereof.

Administering one or more prior dose(s) of the IL-13 binding protein may be continued until the subject reaches a low disease state. For example, if the subject demonstrates an improvement in an AD-associated parameter and/or patient-related outcome, i.e. an IGA score of 0 or 1 and/or a worst Daily Pruritus NRS of <3, following administration of the one or more prior dose(s) of the IL-13 binding protein to the subject for around 12-16 weeks, then the subject is moved to a maintenance phase of treatment, such that the anti-IL-13 antibody, or IL-13-binding fragment thereof, is administered to the subject at a reduced dosing frequency. In this phase, the method comprises administering a first dose of the anti-IL-13 antibody, or IL-13-binding fragment thereof, to the subject; and administering one or more secondary dose(s) of the anti-IL-13 antibody, or IL-13-binding fragment thereof, to the subject, wherein each secondary dose is administered to the subject from 25 days to 31 days, e.g. 28 days, after the immediately preceding dose.

Administering one or more prior dose(s) of the IL-13 binding protein to the subject may be continued for more than 12 weeks, e.g. for about 16 weeks or more, for about 20 weeks or more, for about 24 weeks or more, for about 28 weeks or more, for about 32 weeks or more, for about 36 weeks or more, for about 40 weeks or more, for about 44 weeks or more, for about 48 weeks or more, or for about 52 weeks or more, until the method provides the required improvement in an AD-associated parameter, i.e. IGA score and/or Worst Daily Pruritus NRS, and/or patient-related outcome as described herein. In preferred embodiments, each prior dose is administered to the subject from 12 days to 16 days (e.g. about 2 weeks) after the immediately preceding prior dose. Administering the one or more prior dose(s) from 12 days to 16 days (e.g. about 2 weeks) after the immediately preceding prior dose is continued until the subject achieves an Investigator's Global Assessment (IGA) score of 0 or 1 and/or a Worst Daily Pruritus NRS of <3. Once the subject has achieved the desired response, one or more secondary dose(s) of the IL-13 binding protein can be administered to the subject from 15 days to 35 days after the immediately preceding dose, preferably 28 days after the immediately preceding dose.

Each prior dose may be from about 10 mg to about 600 mg of the anti-IL-13 antibody, or IL-13-binding fragment thereof, from about 50 mg to 500 mg, from 100 mg to about 400 mg, from about 250 mg to about 350 mg or from about 280 mg to about 320 mg of anti-IL-13 antibody, or IL-13-binding fragment thereof. For example, each prior dose is about 10 mg, about 25 mg, about 50 mg, about 100 mg, about 150 mg, about 200 mg, about 250 mg, about 300 mg, about 400 mg or about 500 mg. In some cases, the dose is 600 mg or less, 500 mg or less, 400 mg or less, 300 mg or less, 200 mg or less or 200 mg or less. In some embodiments, each prior dose is about 150 mg of anti-IL-13 antibody, or IL-13-binding fragment thereof. In preferred embodiments, each prior dose is about 300 mg of anti-IL-13 antibody, or IL-13-binding fragment thereof.

When administering one or more prior dose(s) leads to improvement in one or more AD-associated parameter or patient-related outcome as described herein, steps (b) and (c) of the method (i.e. administering a first dose and one or more secondary dose(s) of the anti-IL-13 antibody, or IL-13-binding fragment thereof, to the subject) may further improve, or maintain, the one or more AD-associated parameter or patient-related outcome. When the subject reaches a low disease state by administering one or more prior dose(s), steps (b) and (c) of the method (i.e. administering a first dose and one or more secondary dose(s) of the anti-IL-13 antibody, or IL-13-binding fragment thereof, to the subject) may maintain the low disease state. Preferably, administering a first dose and one or more one or more secondary dose(s) of the anti-IL-13 antibody, or IL-13-binding fragment thereof, may achieve an Investigator's Global Assessment (IGA) score of 0 or 1 and/or ≥75% improvement of Eczema Area and Severity Index (EASI-75) over baseline in a subject.

In some embodiments, each dose (the first dose and one or more secondary dose(s), and optionally the one or more prior dose(s)) is the same amount (in milligrams) of the of the IL-13 binding protein, for example, from about 10 mg to about 600 mg of the anti-IL-13 antibody, or IL-13-binding fragment thereof, from about 50 mg to 500 mg, from about 100 mg to about 400 mg, from about 250 mg to about 350 mg or from about 280 mg to about 320 mg of IL-13 binding protein. In particular, each dose may be about 10 mg, about 25 mg, about 50 mg, about 100 mg, about 150 mg, about 200 mg, about 250 mg, about 300 mg, about 400 mg or about 500 mg. In some cases, the dose is 600 mg or less, 500 mg or less, 400 mg or less, 300 mg or less, 200 mg or less or 200 mg or less. In some embodiments, each dose (i.e. the first dose and one or more secondary dose(s), and optionally the one or more prior dose(s) is about 150 mg of the anti-IL-13 antibody, or IL-13-binding fragment thereof. In preferred embodiments, each dose (i.e. the first dose and one or more secondary dose(s), and optionally the one or more prior dose(s) is about 300 mg of the anti-IL-13 antibody, or IL-13-binding fragment thereof.

In some embodiments, an initial loading dose is administered before said one or more prior doses, wherein the initial loading dose is a bolus dose which is double the amount of the doses following the bolus dose.

In some embodiments the initial loading dose is 600 mg dose and the one or more prior dose(s) following the 600 mg dose are 300 mg dose(s).

In some embodiments a bolus dose is given as an initial loading dose before the above mentioned “one or more prior doses”. The bolus is typically twice the amount of the dose administered with the next administration. For example, a dose of 600 mg is used as a bolus dose when the next dose administered is 300 mg, and a dose of 300 mg is used as a bolus dose when the next dose administered is 150 mg.

### Administration

In the methods described herein, the anti-IL-13 antibody, or IL-13-binding fragment thereof, may be administered by any appropriate method. Typically, administration is parenteral, e.g. intradermal, intramuscular, intravenous and subcutaneous. Subcutaneous administration is particularly preferred. Each dose of the anti-IL-13 antibody, or IL-13-binding fragment thereof, may therefore be administered subcutaneously.

Administration is preferably in a “therapeutically effective amount”, this being sufficient to show improvement or maintained improvement in one or more AD-associated parameter or patient-related outcome as described herein, or achievement of a low disease state.

Administration may be by any convenient route, for example by infusion or bolus injection, by absorption through epithelial or mucocutaneous linings (e.g. oral mucosa, rectal and intestinal mucosa, etc.).

Subcutaneous or intravenous delivery may be with a standard needle and syringe (e.g. including with a prefilled syringe). It is envisaged that the methods described herein will not be restricted to use in the clinic. Therefore, subcutaneous injection using a needle free device is also preferred. Such delivery devices can be reusable or disposable. Numerous reusable pen and autoinjector delivery devices are known in the art and may find use in the present invention. Examples include AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ 1, 11 and 111 (Nova Nordisk, Copenhagen, Denmark), NOVOPEN JUNIOR™ (Nova Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (Sanofi-Aventis, Frankfurt, Germany). Exemplary disposable pen delivery devices for subcutaneous delivery that may find use in the present invention applications include the SOLOSTAR™ pen (Sanofi-Aventis), the FLEXPEN™ (Nova Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L. P.), and the HUMIRA™ Pen (Abbott Labs, Abbott Park IL).

Each dose of anti-IL-13 antibody, or IL-13-binding fragment thereof, is not necessarily administered in a single administration step (e.g. one injection or one tablet etc.). Indeed, depending on the concentration of the anti-IL-13 antibody, or IL-13-binding fragment thereof, (e.g. in the pharmaceutical composition), one, two, three or more administration steps (e.g. one, two, three or more injections) may be required to provide the subject with the required amount anti-IL-13 antibody, or IL-13-binding fragment thereof, (e.g. a 300 mg dose, for example). Thus, in some embodiments, each dose of the anti-IL-13 antibody, or IL-13-binding fragment thereof, is administered in one or two injections (e.g. subcutaneously). Typically subcutaneous injections have a volume of around 1.5 mL or less, such as a volume of from 0.2 to 1.5 mL, e.g. around 1 mL.

### Monotherapy and Combination Therapy

The methods described herein may be a monotherapy. As used herein, the term “monotherapy” is a therapy which uses a single drug to treat a disease or condition. Therefore, a subject that is treated with a monotherapy will receive only a single drug to treat the relevant disorder, e.g. AD or pruritus. For example, an anti-IL-13 antibody monotherapy refers to a monotherapy which comprises the administration of anti-IL-13 antibody, or IL-13-binding fragment thereof, to the subject as the sole drug for the treatment of AD.

Thus, in some embodiments, the anti-IL-13 antibody, or IL-13-binding fragment thereof, is not administered in combination with a topical corticosteroid. “Not administered in combination with” means “not administered in the same course of treatment”. In some embodiments, the anti-IL-13 antibody, or IL-13-binding fragment thereof, is administered as a monotherapy for treating pruritus in a subject having moderate to severe AD.

The methods described herein may be a combination therapy. As used herein, the term “combination therapy” is a therapy which uses more than one drug to treat a disease or condition. For example, a subject that is treated with a combination therapy will receive more than one drug (e.g. two, three or more) to treat AD.

In some embodiments, the anti-IL-13 antibody, or IL-13-binding fragment thereof, is administered in combination with a topical therapy (such as a topical corticosteroid or a topical calcineurin inhibitor). The additional treatment may be administered at any time during the course of treatment with the anti-IL-13 antibody, or IL-13-binding fragment thereof, and for any time period. In some instances, the additional treatment (e.g. TCS or TCI) is administered as needed by the subject.

In some cases, the anti-IL-13 antibody, or IL-13-binding fragment thereof, is administered in combination with a second therapeutic agent selected from the group consisting of a topical corticosteroid, a topical calcineurin inhibitor, an anti-histamine, an emollient, or an anti-bacterial therapeutic. In some cases, the anti-IL-13 antibody, or IL-13-binding fragment thereof, is administered in combination with a Group I, Group II, Group III or Group IV corticosteroid. Preferably, the anti-IL-13 antibody, or IL-13-binding fragment thereof, can be administered in combination with mometasone furoate (e.g. 0.1% cream).

### Pharmaceutical Compositions and Formulations

The present invention envisages methods where each dose of the anti-IL-13 antibody, or IL-13-binding fragment thereof, is administered as a pharmaceutical composition.

The pharmaceutical compositions may be formulated with suitable carriers, excipients, and other agents that provide suitable transfer, delivery, tolerance, and the like. A multitude of formulations can be found in the formulary known to all pharmaceutical chemists: Remington's Pharmaceutical Sciences, Mack Publishing Company, Easton, PA.

The dose administered to a patient according to the methods described herein may be varied depending upon the age and the size of the patient, symptoms, conditions, route of administration, and the like. The dose can be calculated according to body weight or body surface area.

Thus, the pharmaceutical compositions may comprise, in addition to the active ingredient (i.e. the anti-IL-13 antibody, or IL-13-binding fragment thereof), a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. intravenous or subcutaneous.

For intravenous injection or subcutaneous injection, the pharmaceutical composition may be a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

The pharmaceutical composition may be a liquid formulation or a lyophilized formulation which is reconstituted before use. As excipients for a lyophilized formulation, for example, sugar alcohols, or saccharides (e.g. mannitol or glucose) may be used. In the case of a liquid formulation, the pharmaceutical composition is usually provided in the form of containers with defined volume, including sealed and sterilized plastic or glass vials, ampoules and syringes, as well as in the form of large volume containers like bottles. Preferably, in the methods described herein, the pharmaceutical composition is a liquid formulation.

Exemplary pharmaceutical compositions that can be used in the context of the present invention are disclosed in, for example, WO 2007/036745 and WO 2018/158332.

Preferably, the anti-IL-13 antibody, or IL-13-binding fragment thereof, may be present within the pharmaceutical composition at a concentration of from 1 mg/mL to 200 mg/mL, more preferably 150 mg/mL.

Preferably, the anti-IL-13 antibody, or IL-13-binding fragment thereof, may be buffered to a pH of 5.2 to 5.7, most preferably 5.5 (e.g. ±0.1). The selection of such a pH confers significant stability to the pharmaceutical composition. Examples of alternative buffers that control the pH in this range include succinate, gluconate, histidine, citrate, phosphate, glutarate, cacodylate, sodium hydrogen maleate, tris(hydroxymethyl)aminomethane (Tris), 2-(N-morpholino)ethanesulphonic acid (MES), imidazole. Preferably, the buffer is acetate buffer, more preferably sodium acetate buffer.

Preferably, the acetate buffer is present within the pharmaceutical composition in an amount of from 1 mM to 100 mM, more preferably from 30 mM to 70 mM, especially 50 mM.

It will be appreciated that references to “pharmaceutically acceptable excipient” includes references to any excipient conventionally used in pharmaceutical compositions. Such excipients may typically include one or more surfactant, inorganic or organic salt, stabilizer, diluent, solubilizer, reducing agent, antioxidant, chelating agent, preservative and the like.

Examples of a typical surfactant include: nonionic surfactants (HLB 6 to 18) such as sorbitan fatty acid esters (e.g. sorbitan monocaprylate, sorbitan monolaurate, sorbitan monopalmitate), glycerine fatty acid esters (e.g. glycerine monocaprylate, glycerine monomyristate, glycerine monostearate), polyglycerine fatty acid esters (e.g. decaglyceryl monostearate, decaglyceryl distearate, decaglyceryl monolinoleate), polyoxyethylene sorbitan fatty acid esters (e.g. polyoxyethylene sorbitan monolaurate, polyoxyethylene sorbitan monooleate, polyoxyethylene sorbitan monostearate, polyoxyethylene sorbitan monopalmitate, polyoxyethylene sorbitan trioleate, polyoxyethylene sorbitan tristearate), polyoxyethylene sorbitol fatty acid esters (e.g. polyoxyethylene sorbitol tetrastearate, polyoxyethylene sorbitol tetraoleate), polyoxyethylene glycerine fatty acid esters (e.g. polyoxyethylene glyceryl monostearate), polyethylene glycol fatty acid esters (e.g. polyethylene glycol distearate), polyoxyethylene alkyl ethers (e.g. polyoxyethylene lauryl ether), polyoxyethylene polyoxypropylene alkyl ethers (e.g. polyoxyethylene polyoxypropylene glycol ether, polyoxyethylene polyoxypropylene propyl ether, polyoxyethylene polyoxypropylene cetyl ether), polyoxyethylene alkylphenyl ethers (e.g. polyoxyethylene nonylphenyl ether), polyoxyethylene hydrogenated castor oils (e.g. polyoxyethylene castor oil, polyoxyethylene hydrogenated castor oil), polyoxyethylene beeswax derivatives (e.g. polyoxyethylene sorbitol beeswax), polyoxyethylene lanolin derivatives (e.g. polyoxyethylene lanolin), and polyoxyethylene fatty acid amides (e.g. polyoxyethylene stearyl amide); anionic surfactants such as C10-C18 alkyl sulfates salts (e.g. sodium cetyl sulfate, sodium lauryl sulfate, sodium oleyl sulfate), polyoxyethylene C10-C18 alkyl ether sulfates salts with an average of 2 to 4 moles of ethylene oxide (e.g. sodium polyoxyethylene lauryl sulfate), and C8-C18 alkyl sulfosuccinate ester salts (e.g. sodium lauryl sulfosuccinate ester); and natural surfactants such as lecithin, glycerophospholipid, sphingophospholipids (e.g. sphingomyelin), and sucrose esters of C12-C18 fatty acids. The surfactant may be selected from polyoxyethylene sorbitan fatty acid esters. Preferred surfactants are polysorbate 20, 21, 40, 60, 65, 80, 81 and 85, most preferably polysorbate 20 and 80, especially polysorbate 80.

Preferably, the surfactant is present within the pharmaceutical composition in an amount of from 0.001% to 0.1% (w/w), more preferably 0.005% and 0.05% (w/w), especially 0.01% (w/w).

Examples of a typical inorganic salt include: sodium chloride, potassium chloride, calcium chloride, sodium phosphate, sodium sulphate, ammonium sulphate, potassium phosphate and sodium bicarbonate or any other sodium, potassium or calcium salt. Preferably, the inorganic salt is sodium chloride.

Preferably, the inorganic salt is present within the pharmaceutical composition in an amount of from 10 mM to 200 mM, more preferably from 60 mM to 130 mM, especially 85 mM.

Examples of a reducing agent include N-acetylcysteine, Nacetylhomocysteine, thioctic acid, thiodiglycol, thioethanolamine, thioglycerol, thiosorbitol, thioglycolic acid and a salt thereof, sodium thiosulfate, glutathione, and a C1-C7 thioalkanoic acid.

Examples of an antioxidant include erythorbic acid, dibutylhydroxytoluene, butylhydroxyanisole, alpha-tocopherol, tocopherol acetate, L-ascorbic acid and a salt thereof, L-ascorbic acid palmitate, L-ascorbic acid stearate, sodium bisulfite, sodium sulfite, triamyl gallate and propyl gallate.

Examples of a chelating agent include disodium ethylenediaminetetraacetate (EDTA), sodium pyrophosphate and sodium metaphosphate.

Examples of a stabiliser include creatinine, an amino acid selected from histidine, alanine, glutamic acid, glycine, leucine, phenylalanine, methionine, isoleucine, proline, aspartic acid, arginine, lysine and threonine, a carbohydrate selected from sucrose, trehalose, sorbitol, xylitol and mannose, surfactants selected from polyethylene glycol (PEG; e.g. PEG3350 or PEG 4000) or polyoxyethylene sorbitan fatty acid esters (e.g. polysorbate 20 or polysorbate 80), or any combination thereof.

In one preferred embodiment the stabiliser comprises a single carbohydrate (e.g. trehalose).

In an alternatively preferred embodiment the stabilizer comprises an amino acid in combination with a carbohydrate (e.g. trehalose and alanine or trehalose, alanine and glycine).

In a further alternatively preferred embodiment the stabiliser comprises an amino acid in combination with a carbohydrate and a surfactant (e.g. trehalose, alanine and PEG3350; trehalose, proline and PEG3350; trehalose, alanine and polysorbate 80; trehalose, proline and polysorbate 80; trehalose, alanine, glycine and PEG3350; trehalose, alanine, glycine and polysorbate 80).

In a yet further alternatively preferred embodiment the stabiliser comprises an amino acid in combination with a surfactant (e.g. alanine and PEG3350 or alanine, glycine and PEG3350).

In a yet further alternatively preferred embodiment the stabiliser comprises a carbohydrate in combination with a surfactant (e.g. trehalose and PEG3350 or trehalose and polysorbate 80).

Examples of a preservative include octadecyldimethylbenzyl ammonium chloride, hexamethonium chloride, benzalkonium chloride (a mixture of alkylbenzyldimethylammonium chlorides in which the alkyl groups are long chain compounds), benzethonium chloride, aromatic alcohols such as phenol, butyl and benzyl alcohol, alkyl parabens such as methyl or propyl paraben, catechol, resorcinol, cyclohexanol, 3-pentanol, and m-cresol. In a preferred embodiment, the pharmaceutical composition comprises an anti-IL-13 antibody, or IL-13-binding fragment thereof, as described herein, a surfactant and an inorganic salt buffered to a pH of 5.5±0 0.1 with acetate buffer.

In a further preferred embodiment, the pharmaceutical composition comprises an anti-IL-13 antibody, or IL-13-binding fragment thereof, as described herein, sodium chloride and polysorbate 80, buffered to a pH of 5.5±0 0.1 with sodium acetate buffer.

In a yet further preferred embodiment, the pharmaceutical composition comprises an anti-IL-13 antibody, or IL-13-binding fragment thereof, as described herein (e.g. tralokinumab), 50 mM sodium acetate buffer, 85 mM sodium chloride, 0.01% (w/v) polysorbate 80, wherein the pharmaceutical composition has a pH of 5.5.

In a yet further preferred embodiment, the pharmaceutical composition comprises 150 mg/mL of an anti-IL-13 antibody, or IL-13-binding fragment thereof, (e.g. tralokinumab), 50 mM sodium acetate buffer, 85 mM sodium chloride, 0.01% (w/v) polysorbate 80, wherein the pharmaceutical composition has a pH of 5.5.

In another embodiment, the pharmaceutical composition comprises an anti-IL-13 antibody or an IL-13-binding fragment thereof, (e.g. tralokinumab), a histidine buffer, a positively charged excipient, and a surfactant, pH 5.2-5.7.

In one embodiment, the invention provides a pharmaceutical composition comprising an anti-IL-13 antibody, or an IL-13-binding fragment thereof, (e.g. tralokinumab), 20 mM±40% histidine buffer, 150 mM±40% a positively charged excipient (e.g. lysine or arginine), 0.01% surfactant (e.g. polysorbate 80, pH 5.5±0.1).

In another embodiment, the pharmaceutical composition comprises an anti-IL-13 antibody, or an IL-13-binding fragment thereof, (e.g. tralokinumab), at a concentration of between 130 and 170 mg/ml (e.g. 150 mg/ml), 15-25 mM histidine buffer (e.g. 20 mM±10% histidine buffer), 100-200 mM lysine or arginine (e.g. 150 mM±10% lysine or arginine), 0.01% polysorbate 80, pH 5.5±0.1.

### Other Definitions

Unless defined otherwise, all technical and scientific terms used herein have the same meaning as commonly understood to one of ordinary skill in the art to which this invention belongs.

The articles “a” and “an”” refer to one or to more than one (i.e., to at least one) of the grammatical object of the article. By way of example, “an element” means one element or more than one element.

The terms “comprise” and “comprising” are used in the inclusive, open sense, meaning that additional elements may be included.

In general, methods “comprising” a number of steps do not require the steps to be performed in a particular order. Where a method comprises a number of sequentially numbered or alphabetical steps (e.g. (1), (2), (3); (i), (ii), (iii); or (a), (b), (c) etc.), this implies that the steps must be performed in the prescribed order unless stated otherwise.

The term “including” is used herein to mean “including but not limited to”.

The term “about” in relation to a numerical value x is optional and means, for example, x±10%.

Generally, the terms “treat”, “treating”, “treatment”, or the like, mean to alleviate (reduce, minimise, or eliminate) symptoms, or to reduce, minimise or eliminate the causation of symptoms either on a temporary or permanent basis. All publications mentioned herein are incorporated by reference in their entirety.

As used herein, the term “sequence identity” or “identity” denotes a property of sequences that measures their similarity or relationship. The term “sequence identity” or “identity” as used in the present disclosure means the percentage of pair-wise identical residues—following (homologous) alignment of a sequence of a protein or polypeptide of the disclosure with a sequence in question—with respect to the number of residues in the longer of these two sequences. Sequence identity is measured by dividing the number of identical amino acid residues by the total number of residues and multiplying the product by 100.

A skilled artisan will recognize available computer programs, for example BLAST (Altschul et al., Nucleic Acids Res, 1997), BLAST2 (Altschul et al., J Mol Biol, 1990), FASTA (which uses the method of Pearson and Lipman (1988)), the TBLASTN program, of Altschul et al. (1990) supra, GAP (Wisconsin GCG package, Accelerys Inc, San Diego USA) and Smith-Waterman (Smith and Waterman, J Mol Biol, 1981), for determining sequence identity using standard parameters. The percentage of sequence identity can, for example, be determined herein using the program BLASTP, version 2.2.5, Nov. 16, 2002 (Altschul et al., Nucleic Acids Res, 1997). In this embodiment, the percentage of homology is based on the alignment of the entire protein or polypeptide sequences (matrix: BLOSUM 62; gap costs: 11.1; cut off value set to 10⁻³) including the polypeptide sequences, preferably using the wild-type protein scaffold as reference in a pairwise comparison. It is calculated as the percentage of numbers of “positives” (homologous amino acids) indicated as result in the BLASTP program output divided by the total number of amino acids selected by the program for the alignment. Sequence identity is commonly defined with reference to the algorithm GAP (Wisconsin GCG package, Accelerys Inc, San Diego USA). GAP uses the Needleman and Wunsch algorithm to align two complete sequences, maximising the number of matches and minimising the number of gaps, which are spaces in an alignment that are the result of additions or deletions of amino acids. Generally, default parameters are used, with a gap creation penalty equalling 12 and a gap extension penalty equalling 4.

Specifically, in order to determine whether an amino acid residue of the amino acid sequence of an anti-IL-13 antibody or a fragment thereof is different from another antibody sequence, a skilled artisan can use means and methods well-known in the art, e.g., alignments, either manually or by using computer programs such as BLAST 2.0, which stands for Basic Local Alignment Search Tool, or ClustalW, or any other suitable program which is suitable to generate sequence alignments.

### Examples

The invention is further illustrated by the following examples. It will be appreciated that the examples are for illustrative purposes only and are not intended to limit the invention as described above. Modification of detail may be made without departing from the scope of the invention.

### Introduction & Objectives

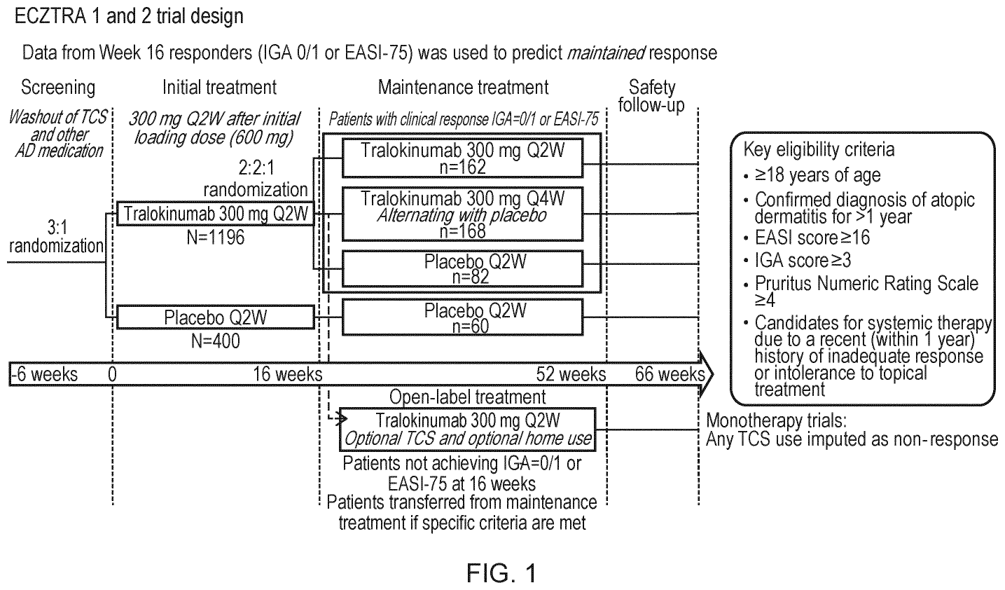

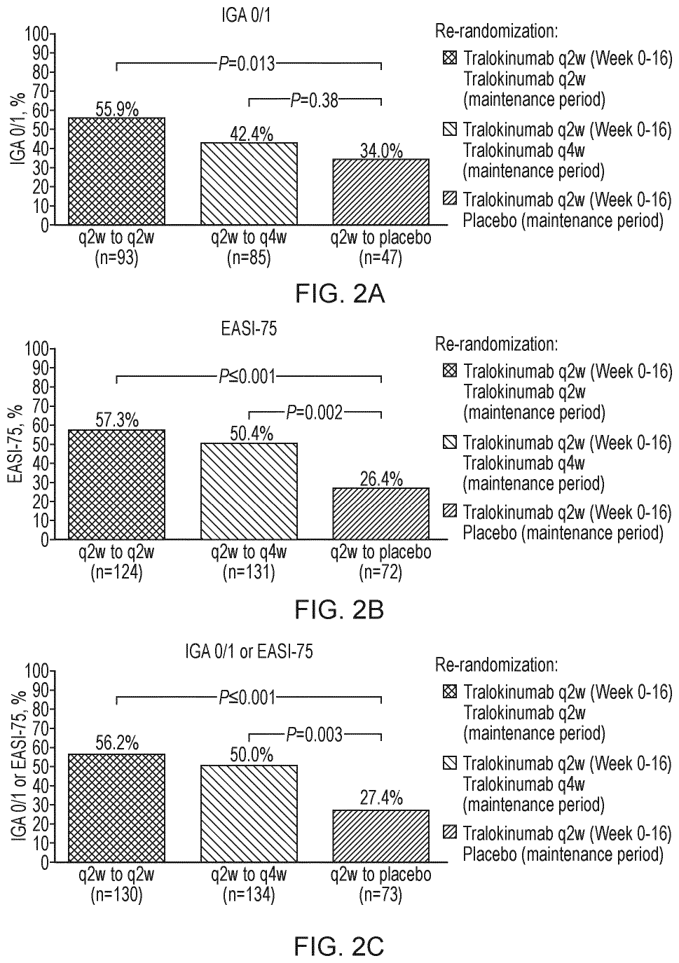

Tralokinumab, a high-affinity, monoclonal antibody that specifically neutralizes IL-13, was assessed in two Phase 3 trials (ECZTRA 1 and 2) of moderate-to-severe atopic dermatitis (Wollenberg, A., et al., Tralokinumab for moderate-to-severe atopic dermatitis: results from two 52-week, randomized, double-blind, multicentre, placebo-controlled phase III trials (ECZTRA 1 and ECZTRA 2); Br J Dermatol, 2021. 184(3): p. 437-449). Tralokinumab 300 mg every two weeks (Q2W), after a 600 mg loading dose, demonstrated superiority to placebo for primary and secondary endpoints at Week (Wk) 16. Patients that met primary endpoints, Investigator's Global Assessment (IGA) 0/1 and/or Eczema Area and Severity Index (EASI)-75, were re-randomized to tralokinumab Q2W, every four weeks (Q4W), or placebo for another 36 weeks (see FIG. 1). At week 52, IGA 0/1 or EASI-75 was maintained without any rescue use (including TCS) in a majority of patients on the Q2W dosing regimen, but the ability to maintain clear or almost clear skin at week 52 was reduced to around 40% in patients achieving clear or almost clear skin at week 16 and then subsequently re-randomised to Q4W dosing regimen (see FIG. 2). At Wk 52, IGA 0/1 or EASI-75 was maintained without rescue use (including TCS) in 56.2% (73/130; P<0.001), 50.0% (67/134; P=0.003), and 27.4% (20/73) in the Q2W, Q4W, and placebo arms, respectively (see FIG. 2C). These data showed that continuing with tralokinumab Q2W or Q4W led to greater maintenance of response at Week 52 relative to placebo. Here potential early predictors of maintained response at Wk 52 with tralokinumab Q4W versus Q2W dosing were evaluated.

### Methods

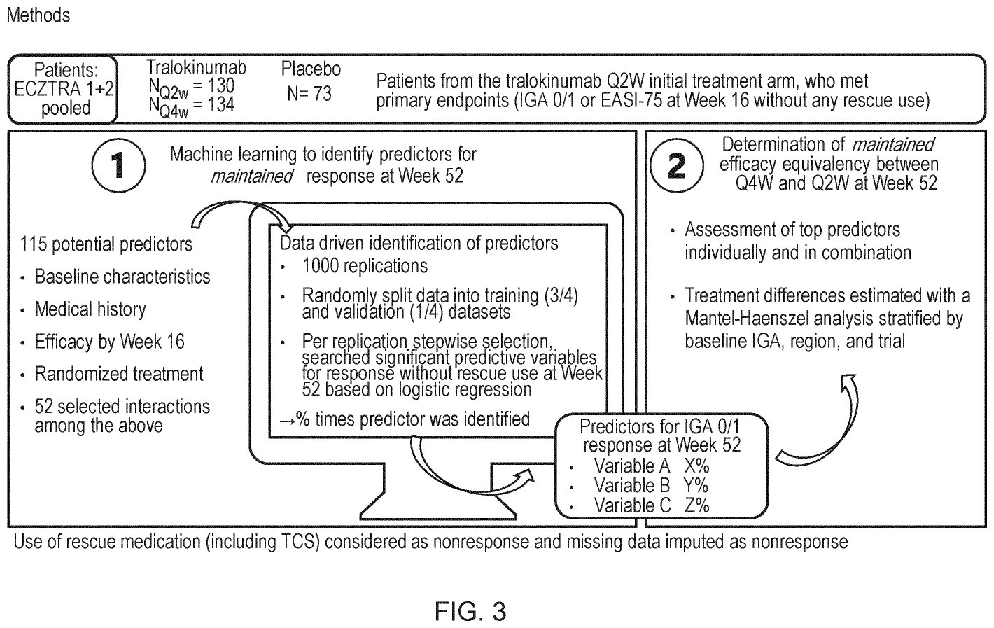

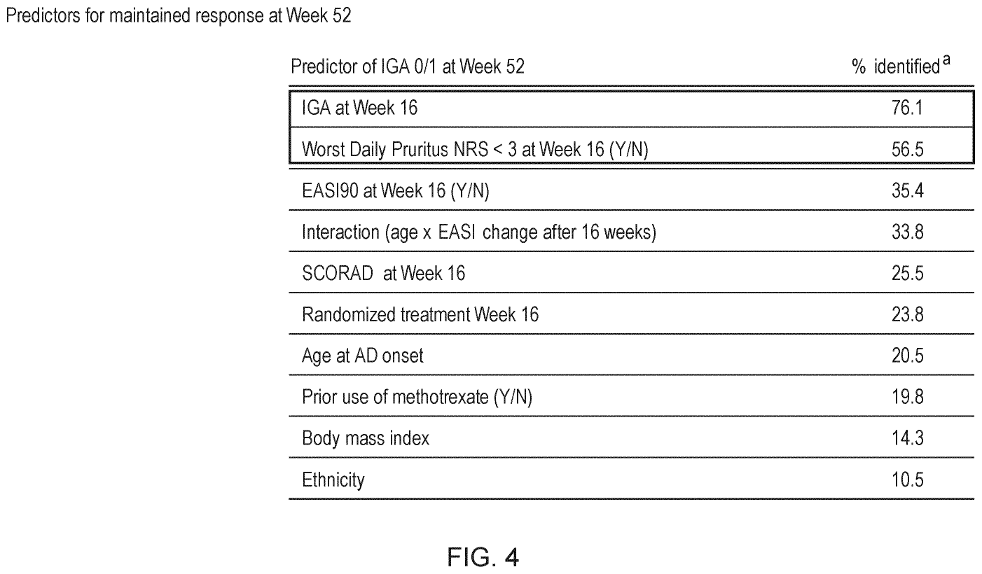

Patients who met primary endpoints at Wk 16 (N=337) were included in post hoc analyses using a machine learning algorithm to identify predictors for maintained response at Wk 52. Potential predictors included demographic, medical/medication history, baseline efficacy, efficacy after 16 weeks or earlier and randomized treatment (63 main factors, 52 interactions). The algorithm utilized 1000 replications for which data was randomly split into training (75% of data) and validation (25%) datasets. Per replication stepwise selection was used to systematically search combinations of significant (P<0.05) predictive variables for IGA 0/1 without rescue use at Wk 52 based on logistic regression (see FIG. 3). These predictors were ranked by percentage of times identified by the algorithm in the final predictive model for IGA 0/1 at Wk 52; a higher value indicates greater predictive ability (see FIG. 4). Top predictors were then assessed individually and in combination to determine maintained efficacy equivalency between Q4W and Q2W at Wk 52. Treatment differences were estimated with a Mantel-Haenszel analysis stratified by baseline IGA, region, and trial.

### Results

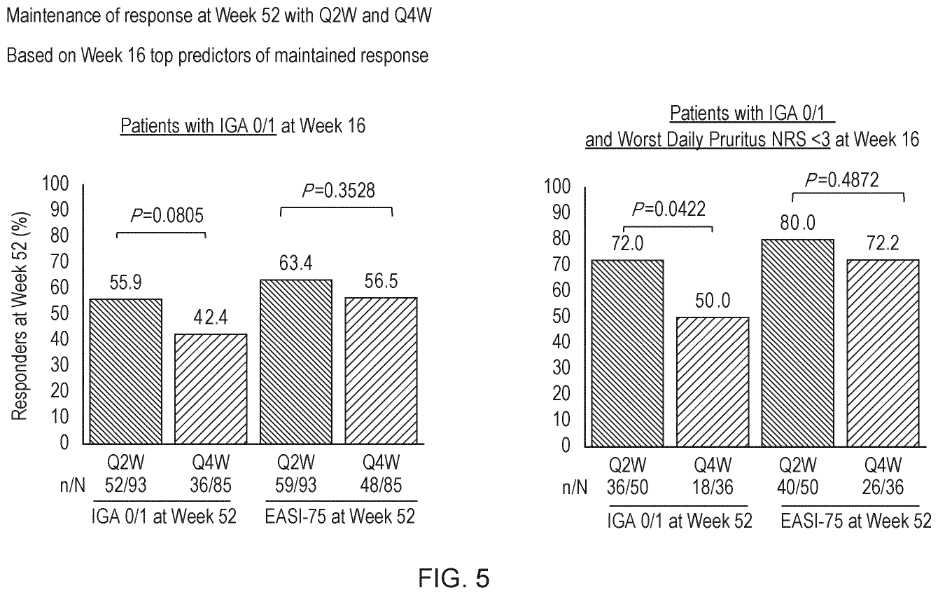

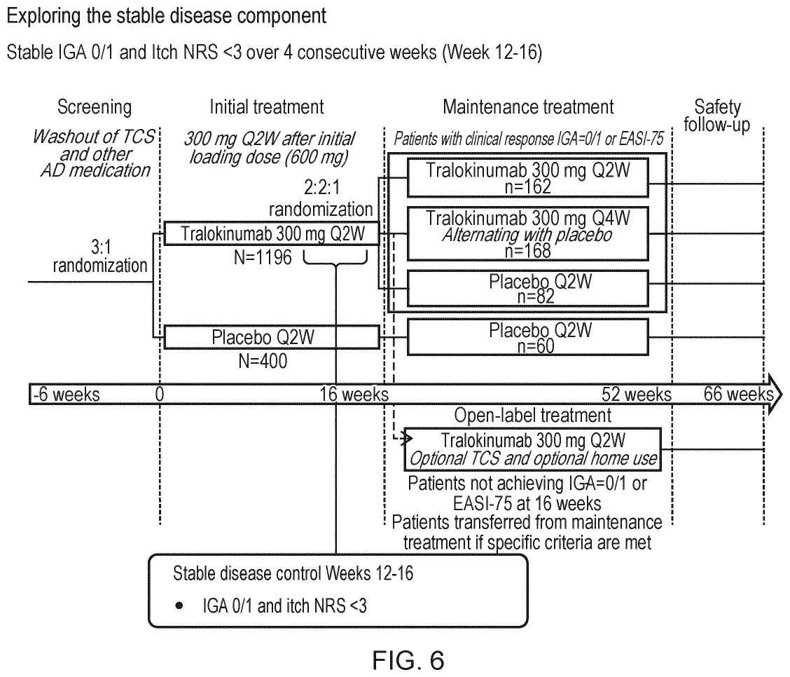

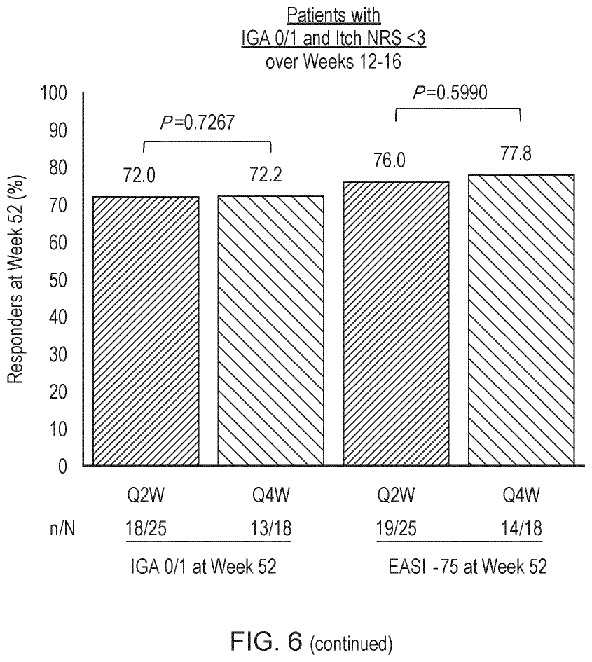

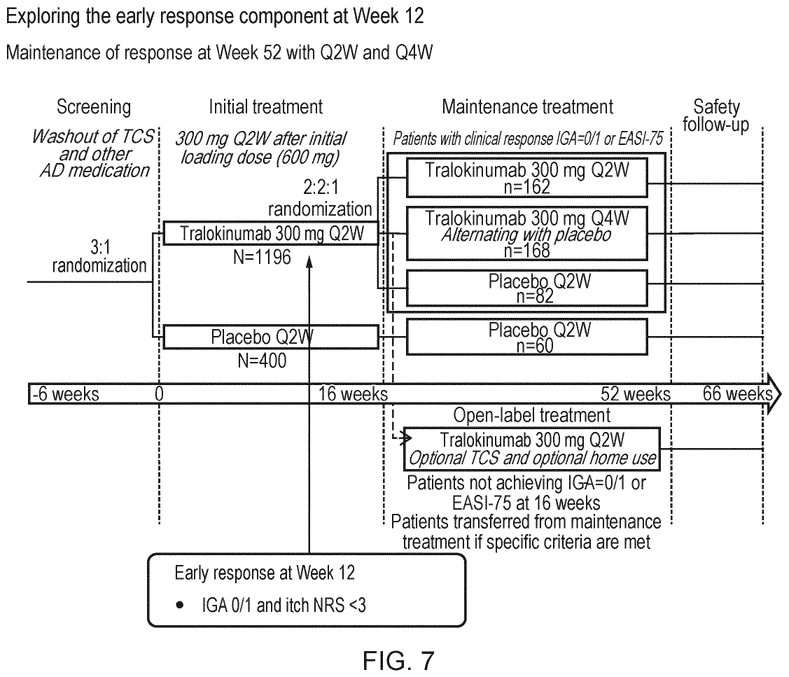

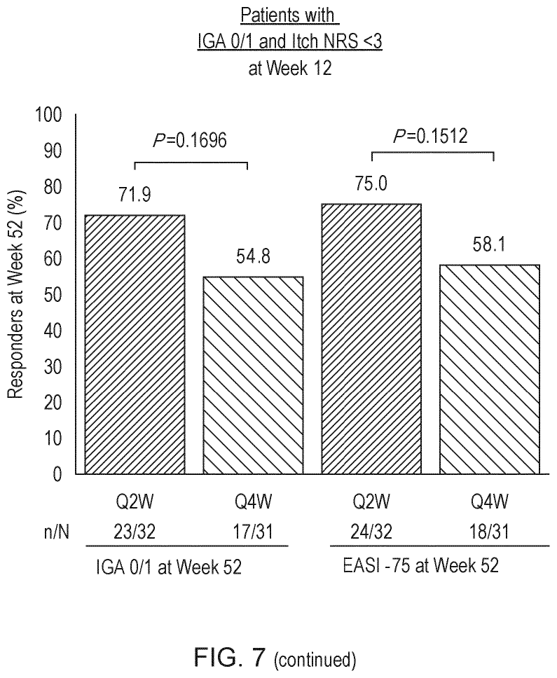

The top two predictors for maintaining IGA 0/1 at Wk 52 regardless of treatment dose frequency were IGA score (identified 76.1% of times) and Worst Daily Pruritus Numerical Rating Scale (NRS)<3 (56.6%) at Wk 16 (see FIG. 4). When assessing efficacy equivalency between Q2W and Q4W, a numerically higher proportion of patients with IGA 0/1 at Wk 16 maintained response with Q2W relative to Q4W at Wk 52 (see FIG. 5). Rates of maintained response at Wk 52 were higher in patients with both of the top two predictors at Wk 16 relative to those with IGA 0/1 only (see FIG. 5). For patients who met both top two predictors at Wks 12/14/16 (4 weeks stable response) Q2W and Q4W maintained response comparably well at Wk 52 (see FIG. 6); similar equivalence between Q2W and Q4W for these patients was observed for EASI-75 and in the ECZTRA 3 trial (Silverberg, J. I. et al., Tralokinumab plus topical corticosteroids for the treatment of moderate-to-severe atopic dermatitis: results from the double-blind, randomized, multicentre, placebo-controlled phase III ECZTRA 3 trial. Br J Dermatol, 2021. 184(3): p. 450-463). For patients that achieved IGA 0/1 and Itch NRS<3 at week 12, analysis showed that an early response at week 12 increases the likelihood of maintaining response at week 52 for both dosing regimes (see FIG. 7).

### Conclusions

Regardless of treatment dose frequency, IGA score and Worst Daily Pruritus NRS<3 at Wk 16 were the top two predictors of maintained treatment response at Wk 52 with tralokinumab monotherapy. Stable achievement at consecutive timepoints of clear or almost clear skin and mild or no itch symptoms with tralokinumab Q2W over 4 consecutive weeks was identified as a positive predictor of maintained long-term response with the Q4W dosing regimen.

### LIST OF ABBREVIATIONS

AD, atopic dermatitis
DLQI, Dermatology Life Quality Index
EASI, Eczema Area and Severity Index
EASI-50, at least 50% reduction in Eczema Area and Severity Index score
EASI-75, at least 75% reduction in Eczema Area and Severity Index score
EASI-90, at least 90% reduction in Eczema Area and Severity Index score
IGA, Investigator's Global Assessment
IGA-0/1, Investigators' Global Assessment score of 0 (clear) or 1 (almost clear)
NRS, Numeric Rating Scale
PYE, patient-years of exposure
Q2W, every other week, i.e. every 2 weeks
Q4W, every 4 weeks
R, rate (number of AEs divided by PYE multiplied by 100)
SCORAD, SCORing Atopic Dermatitis
TCS, topical corticosteroids

1. An anti-interleukin-13 (IL-13) antibody, or an IL-13-binding fragment thereof, for use in a method of treating atopic dermatitis (AD) in a subject, wherein the method comprises the steps of:
(a) administering one or more prior dose(s) of the anti-IL-13 antibody, or IL-13-binding fragment thereof, to the subject, wherein each prior dose is administered from 12 days to 16 days after the immediately preceding dose, until the subject achieves an Investigator's Global Assessment (IGA) score of 0 or 1 and a worst Daily Pruritus Numeric Rating Scale (NRS) of <3 (i.e. 0-2);
(b) administering a first dose of the anti-IL-13 antibody, or IL-13-binding fragment thereof, to the subject; and
(c) administering one or more secondary dose(s) of the anti-IL-13 antibody, or IL-13-binding fragment thereof, to the subject, wherein each secondary dose is administered to the subject from 25 days to 31 days after the immediately preceding dose.

2. A method of treating atopic dermatitis (AD) in a subject in need thereof, wherein the method comprises the steps of:
(a) administering one or more prior dose(s) of the anti-IL-13 antibody, or IL-13-binding fragment thereof, to the subject, wherein each prior dose is administered from 12 days to 16 days after the immediately preceding dose, until the subject achieves an Investigator's Global Assessment (IGA) score of 0 or 1 and a worst Daily Pruritus Numeric Rating Scale (NRS) of <3;
(b) administering a first dose of the anti-IL-13 antibody, or IL-13-binding fragment thereof, to the subject; and
(c) administering one or more secondary dose(s) of the anti-IL-13 antibody, or IL-13-binding fragment thereof, to the subject, wherein each secondary dose is administered to the subject from 25 days to 31 days after the immediately preceding dose.

3. Use of an anti-IL-13 antibody, or an IL-13-binding fragment thereof, in the manufacture of a medicament for treating atopic dermatitis (AD) in a subject, wherein the method for treating AD comprises the steps of:
(a) administering one or more prior dose(s) of the anti-IL-13 antibody, or IL-13-binding fragment thereof, to the subject, wherein each prior dose is administered from 12 days to 16 days after the immediately preceding dose, until the subject achieves an Investigator's Global Assessment (IGA) score of 0 or 1 and a worst Daily Pruritus Numeric Rating Scale (NRS) of <3;
(b) administering a first dose of the anti-IL-13 antibody, or IL-13-binding fragment thereof, to the subject; and
(c) administering one or more secondary dose(s) of the anti-IL-13 antibody, or IL-13-binding fragment thereof, to the subject, wherein each secondary dose is administered to the subject from 25 days to 31 days after the immediately preceding dose.

4. The anti-IL-13 antibody, or IL-13-binding fragment thereof, for use, the method or the use of any one of the preceding claims, wherein each prior dose of the anti-IL-13 antibody, or IL-13-binding fragment thereof, is administered about 2 weeks after the immediately preceding dose.

5. The anti-IL-13 antibody, or IL-13-binding fragment thereof, for use, the method or the use of any one of the preceding claims, wherein each prior dose of the anti-IL-13 antibody, or IL-13-binding fragment thereof, is administered 14 days after the immediately preceding dose.

6. The anti-IL-13 antibody, or IL-13-binding fragment thereof, for use, the method or the use of any one of the preceding claims, wherein each secondary dose of the anti-IL-13 antibody, or IL-13-binding fragment thereof, is administered about 4 weeks after the immediately preceding dose.

7. The anti-IL-13 antibody, or IL-13-binding fragment thereof, for use, the method or the use of any one of the preceding claims, wherein each secondary dose of the anti-IL-13 antibody, or IL-13-binding fragment thereof, is administered 28 days after the immediately preceding dose.

8. The anti-IL-13 antibody, or IL-13-binding fragment thereof, for use, the method or the use of any one of the preceding claims, wherein the subject achieves an IGA score of 0 or 1 and a Worst Daily Pruritus NRS of <3 (i.e. 0-2) for at least one week, at least two consecutive weeks, at least three consecutive weeks or at least four consecutive weeks.

9. The anti-IL-13 antibody, or IL-13-binding fragment thereof, for use, of any one of claims 1-7, wherein the subject achieves a mean IGA score of 0 or 1 and a mean Worst Daily Pruritus NRS of <3 (i.e. 0-2) over a one week, two week, three week or four week period.

10. The anti-IL-13 antibody, or IL-13-binding fragment thereof, for use, the method or the use of any one of the preceding claims, wherein the method comprises administering the one or more prior doses(s) of an anti-IL-13 antibody, or an IL-13-binding fragment thereof, to the subject for from 2 to 36 weeks.

11. The anti-IL-13 antibody, or IL-13-binding fragment thereof, for use, the method or the use of any one of the preceding claims, wherein the method comprises administering the one or more prior doses(s) of an anti-IL-13 antibody, or an IL-13-binding fragment thereof, to the subject for from about 12 to about 16 weeks.

12. The anti-IL-13 antibody, or IL-13-binding fragment thereof, for use, the method or the use of any one of the preceding claims, wherein the method comprises administering the one or more prior doses(s) of an anti-IL-13 antibody, or an IL-13-binding fragment thereof, to the subject for about 12 weeks, for about 13 weeks, for about 14 weeks, for about 15 weeks or for about 16 weeks (e.g. for about 12 weeks or for about 16 weeks).

13. The anti-IL-13 antibody, or IL-13-binding fragment thereof, for use, the method or the use of any one of the preceding claims, wherein the step of administering one or more secondary dose(s) of the anti-IL-13 antibody, or IL-13-binding fragment thereof, to the subject is continued for at least 8 weeks, at least 12 weeks, at least 16 weeks, at least 20 weeks, at least 24 weeks, at least 28 weeks, at least 32 weeks, at least 36 weeks, at least 40 weeks, at least 44 weeks, at least 48 weeks, or at least 52 weeks.

14. The anti-IL-13 antibody, or IL-13-binding fragment thereof, for use, the method or the use of any one of the preceding claims, wherein an initial loading dose of the anti-IL-13 antibody, or IL-13-binding fragment thereof, is administered to the subject before administering the one or more prior doses.

15. The anti-IL-13 antibody, or IL-13-binding fragment thereof, for use, the method or the use of any one of the preceding claims, wherein each prior dose of the anti-IL-13 antibody, or IL-13-binding fragment thereof, is 250-350 mg.

16. The anti-IL-13 antibody, or IL-13-binding fragment thereof, for use, the method or the use of any one of the preceding claims, wherein each prior dose of the anti-IL-13 antibody, or IL-13-binding fragment thereof, is 300 mg.

17. The anti-IL-13 antibody, or IL-13-binding fragment thereof, for use, the method or the use of any one of the preceding claims, wherein each secondary dose of the anti-IL-13 antibody, or IL-13-binding fragment thereof, is 250-350 mg.

18. The anti-IL-13 antibody, or IL-13-binding fragment thereof, for use, the method or the use of any one of the preceding claims, wherein the first dose and each secondary dose of the anti-IL-13 antibody, or IL-13-binding fragment thereof, is 300 mg.

19. The anti-IL-13 antibody, or IL-13-binding fragment thereof, for use, the method or the use of any one of the preceding claims, wherein the initial loading dose of the anti-IL-13 antibody, or IL-13-binding fragment thereof, is 600 mg.

20. The anti-IL-13 antibody, or IL-13-binding fragment thereof, for use, the method or the use of any one of the preceding claims, wherein each dose of the anti-IL-13 antibody, or IL-13-binding fragment thereof, is administered subcutaneously.

21. The anti-IL-13 antibody, or IL-13-binding fragment thereof, for use, the method or the use of any one of the preceding claims, wherein each dose of the anti-IL-13 antibody, or IL-13-binding fragment thereof, is administered in one or two injections.

22. The anti-IL-13 antibody, or IL-13-binding fragment thereof, for use, the method or the use of any one of the preceding claims, wherein the anti-IL-13 antibody, or IL-13-binding fragment thereof, is administered as a monotherapy.

23. The anti-IL-13 antibody, or IL-13-binding fragment thereof, for use, the method or the use of any one of the preceding claims, wherein the anti-IL-13 antibody, or IL-13-binding fragment thereof, is administered with a second agent, e.g. a topical corticosteroid, a topical calcineurin inhibitor, an anti-histamine, an emollient, or an anti-bacterial therapeutic.

24. The anti-IL-13 antibody, or IL-13-binding fragment thereof, for use, the method or the use of any one of the preceding claims, wherein the method achieves:
(i) ≥75% improvement of Eczema Area and Severity Index (EASI-75) compared to baseline; and/or
(ii) an IGA score of 0 or 1
after 12 weeks, after 16 weeks, after 20 weeks, after 24 weeks, after 28 weeks, after 32 weeks, after 36 weeks, after 40 weeks, after 44 weeks, after 48 weeks, or after 52 weeks.

25. The anti-IL-13 antibody, or IL-13-binding fragment thereof, for use, the method or the use of any one of the preceding claims, wherein the AD is moderate-to-severe or severe AD.

26. The IL-13 binding protein for use, the method or the use according to any one of the preceding claims, wherein each dose of the anti-IL-13 antibody, or IL-13-binding fragment thereof, is administered as a pharmaceutical composition comprising 50 mM sodium acetate buffer, 85 mM sodium chloride, 0.01% (w/v) polysorbate 80, wherein the pharmaceutical composition has a pH of 5.5.

27. The anti-IL-13 antibody, or IL-13-binding fragment thereof, for use, the method or the use according to any one of the preceding claims, wherein the IL-13 binding protein is a monoclonal anti-IL-13 antibody, or an IL-13-binding fragment thereof.

28. The anti-IL-13 antibody, or IL-13-binding fragment thereof, for use, the method or the use according to any one of the preceding claims, wherein the anti-IL-13 antibody, or IL-13-binding fragment thereof, is a human anti-IL-13 antibody, or an IL-13-binding fragment thereof.

29. The anti-IL-13 antibody, or IL-13-binding fragment thereof, for use, the method or the use according to any one of the preceding claims, wherein the IL-13 antibody is an IgG4 antibody.

30. The anti-IL-13 antibody, or IL-13-binding fragment thereof, for use, the method or the use according to any one of the preceding claims, wherein the IL-13-binding fragment is selected from a Fab, Fab′, F(ab′)2, Fd, Fv, single-chain Fv (scFv), or disulfide-linked Fvs (sdFv).

31. The anti-IL-13 antibody, or IL-13-binding fragment thereof, for use, the method or the use according to any one of the preceding claims, wherein the anti-IL-13 antibody, or the IL-13-binding fragment thereof, comprises a heavy chain variable region (HCVR) and a light chain variable region (LCVR), wherein:
(i) the heavy chain variable region comprises:
a heavy chain complementarity determining region 1 (HCDR1) comprising an amino acid sequence of SEQ ID NO:1;
a heavy chain complementarity determining region 2 (HCDR2) comprising an amino acid sequence of SEQ ID NO:2; and
a heavy chain complementarity determining region 3 (HCDR3) comprising an amino acid sequence of SEQ ID NO:3; and
(ii) the light chain variable region comprises:
a light chain complementarity determining region 1 (LCDR1) comprising an amino acid sequence of SEQ ID NO:4;
a light chain complementarity determining region 2 (LCDR2) comprising an amino acid sequence of SEQ ID NO:5; and
a light chain complementarity determining region 3 (LCDR3) comprising an amino acid sequence of SEQ ID NO:6.

32. The anti-IL-13 antibody, or IL-13-binding fragment thereof, for use, the method or the use according to any one of the preceding claims, wherein the anti-IL-13 antibody, or the IL-13-binding fragment thereof, further comprises:
(i) an amino acid sequence that is at least 80%, at least 85%, at least 90%, at least 95%, at least 96%, at least 97%, at least 98%, or at least 99% identical to a heavy chain variable region sequence of SEQ ID NO: 8; and/or
(ii) an amino acid sequence that is at least 80%, at least 85%, at least 90%, at least 95%, at least 96%, at least 97%, at least 98%, or at least 99% identical to a light chain variable region sequence of SEQ ID NO: 10.

33. The anti-IL-13 antibody, or IL-13-binding fragment thereof, for use, the method or the use according to any one of the preceding claims, wherein the anti-IL-13 antibody, or the IL-13-binding fragment thereof, comprises a heavy chain variable region sequence of SEQ ID NO: 8 and a light chain variable region sequence of SEQ ID NO: 10.

34. The anti-IL-13 antibody, or IL-13-binding fragment thereof, for use, the method or the use according to any one of the preceding claims, wherein the anti-IL-13 antibody, or the IL-13-binding fragment thereof, comprises: (i) an amino acid sequence that is at least 80%, at least 85%, at least 90%, at least 95%, at least 96%, at least 97%, at least 98%, or at least 99% identical to the heavy chain sequence of SEQ ID NO: 11; and/or (ii) an amino acid sequence that is at least 80%, at least 85%, at least 90%, at least 95%, at least 96%, at least 97%, at least 98%, or at least 99% identical to the light chain sequence of SEQ ID NO: 12.

35. The anti-IL-13 antibody, or IL-13-binding fragment thereof, for use, the method or the use according to any one of the preceding claims, wherein the anti-IL-13 antibody, or the IL-13-binding fragment thereof, comprises a heavy chain of SEQ ID NO: 11 and a light chain sequence of SEQ ID NO: 12.

36. The anti-IL-13 antibody, or IL-13-binding fragment thereof, for use, the method or the use according to any one of the preceding claims, wherein the anti-IL-13 antibody is Tralokinumab or an IL-13 binding fragment thereof.
